(19) United States
(12) Patent Application Publication
(10) Pub. No.: US 2024/0183857 A1
(43) Pub. Date: Jun. 6, 2024

(54) Protein Tyrosine Phosphatases as Biomarkers for Hepatocellular Carcinoma and Uses Thereof

(71) Applicant: Université Libre De Bruxelles, Brussels (BE)

(72) Inventors: Esteban Gurzov, Bruxelles (BE); Wei Wu, Utrecht (NL)

(21) Appl. No.: 18/553,044

(22) PCT Filed: Mar. 29, 2022
(86) PCT No.: PCT/EP2022/058188

§ 371 Date: Sep. 28, 2023

(30) Foreign Application Priority Data
Mar. 29, 2021 (EP) 21165444.7

(51) Int. Cl. G01N 33/68

(52) U.S. Cl. CPC G01N 33/68; G01N 2333/916; G01N 2800/50; G01N 2800/52

(57) ABSTRACT

This application discloses in vitro methods for the diagnosis, prognosis, and/or monitoring of cancer, in particular liver cancer or hepatocellular carcinoma (HCC) based on the aggregate expression level and oxidation level of all protein tyrosine phosphatases in a sample; whether or not in combination with the differential expression of protein tyrosine phosphatases individually. Also provided are in vitro methods for the selection of a prophylactic or therapeutic treatment or the evaluate the efficacy of a therapeutic treatment. Further, the present application provides a kit to be used in said methods.

### CROSS-REFERENCE TO RELATED APPLICATIONS

This application is a national phase entry under 35 U.S.C. § 371 of International Patent Application PCT/EP2022/058188, filed Mar. 29, 2022, designating the United States of America and published in English as International Patent Publication WO 2022/207588 on Oct. 6, 2022, which claims the benefit under Article 8 of the Patent Cooperation Treaty to European Patent Application Serial No. 21165444.7, filed Mar. 29, 2021, the entireties of which are hereby incorporated by reference.

### FIELD OF THE INVENTION

The invention is broadly in the medical field, particularly in the fields of diagnostic and prognostic testing, and personalised medicine. The invention relates to biomarkers useful for the evaluation of hepatocellular carcinoma (HCC) in subjects, and to related methods, uses, kits and therapeutic or prophylactic agents.

### BACKGROUND OF THE INVENTION

Liver cancer is the fourth most common cause of cancer death worldwide. Hepatocellular carcinoma (HCC) accounts for 90% of primary liver cancers and is refractory to nearly all currently available anti-cancer therapies with a 5-year survival rate of nearly 15%. Over the last 20 years, the incidence of HCC has been rapidly increasing in economically developed nations and is mostly attributable to hepatitis C virus (HCV), alcoholic, and non-alcoholic fatty liver disease (NAFLD). With the development of novel antiviral therapies against HCV, the obesity epidemic is thought to now account for as much as 40% of the increase in HCC in developed countries. Liver cancer is the 10^{th} most frequent cause of death from cancer in males and the 8^{th} in females in Belgium (Belgian Cancer Registry). The hallmark of NAFLD is the accumulation of fat in hepatocytes. Indeed, the main risk factors of NAFLD are obesity and type 2 diabetes (steatosis occurs in >75% of all obese individuals and prevalence increases by a factor 4.6 if BMI≥30 kg/m²) which can progress to fibrosis, cirrhosis, non-alcoholic steatohepatitis (NASH) and HCC. There are ˜1.6 billion overweight adults [BMI>25 kg/m²] worldwide of whom >650 million are obese (BMI>30 kg/m²). This is predicted to rise in the near future and to be largely unabated by lifestyle intervention (WHO). Obesity is associated with a state of chronic low-grade inflammation.

With an increasing number of patients developing NASH-related end-stage liver disease and pharmacological treatments on the horizon, there is a pressing need to develop HCC biomarkers for prognostication, selection of patients for treatment and monitoring, in addition to the need for biomarkers for HCC itself.

Protein tyrosine phosphatases (PTPs) were initially described as a small family of ubiquitously expressed enzymes with generalized housekeeping roles. They are responsible for the modulation of protein tyrosine kinase (PTK) activity through dephosphorylation of various tyrosine residues on intracellular proteins, thus regulating fundamental cellular events such as ligand mediated receptor signalling and cell cycle events. The classical PTPs are subdivided into receptor-like (21 members) and non-transmembrane (17 members). Although initially overlooked, it now becomes clear that PTP actions might have implication in modulation of cellular function, which became apparent as the importance of stringent protein phosphorylation levels in signalling events to maintain cellular homeostasis was realised. Loss of this tight regulation in protein phosphorylation results in over- and under-activation of key signalling pathways that are implicated in the pathogenesis of various diseases, including obesity, steatosis and HCC (Tonks, FEBS J. 2013; Gurzov et al., Trens Endocrinol Metb 2015). Most of the recent studies and available technologies only focus on the role of the individual PTP members, the oxidation profile of PTPs in cell lines and zebrafish (Wu et al., 2017, Sci Rep; Karisch et al., Cell 2011), and the effects of their inactivation by ROS for example in liver diseases such as liver steatosis, NAFD, NASH and HCC, as for example described in Gurzov et al. (Cell Metabolism 2014), Grohmann et al. (Cell, 2018) and Litwak et al. (Diabetes, 2017).

### SUMMARY

Present inventors developed a novel risk diagnostic and/or prognostic tool based on the aggregate expression level of protein tyrosine phosphatases (PTPs) and their aggregate oxidation level, whether or not in combination with their differential expression profile and activity. More specifically, the inventors found that the aggregate expression level of PTPs in a sample in combination with their aggregate oxidation level and optionally in combination with their differential expression allows the diagnosis, prognosis and/or monitoring of cancer, more specifically of hepatocellular carcinoma (HCC) in a human subject.

Therefore, in a first aspect, the present application provides an in vitro method for the diagnosis, prognosis and/or monitoring of cancer in a subject, said method comprising determining the aggregate expression level of PTPs in a sample of the subject, determining the aggregate oxidation level of PTPs in the sample and comparing said aggregate expression level and said aggregate oxidation level of PTPs to a corresponding reference value or threshold value for the aggregate expression and oxidation level of PTPs that is characteristic of a subject with a known diagnosis, prognosis and/or disease status of cancer, wherein a deviation or no deviation is indicative for the diagnosis, prognosis and/or disease status of the subject.

In another aspect, an in vitro method for determining the risk of developing cancer in a subject is disclosed, said method comprising determining the aggregate expression level of PTPs in a sample of the subject, determining the aggregate oxidation level of PTPs in the sample, and comparing said aggregate expression and oxidation level of PTPs to a corresponding reference value or threshold value for the aggregate expression and oxidation level of PTPs that is characteristic of a subject with a known diagnosis, prognosis and/or disease status of cancer, wherein a deviation or no deviation is indicative for the risk of developing cancer in the subject.

In a further aspect, an in vitro method for assisting in the selection of a prophylactic or therapeutic 4 0 & treatment of cancer in a subject is disclosed, said method comprising determining the aggregate expression level of PTPs in a sample of the subject, determining the aggregate oxidation level of PTPs in the sample, and comparing said aggregate expression and oxidation level of PTPs to a corresponding reference value or threshold value for the aggregate expression and oxidation level of PTPs that is characteristic of a subject with a known diagnosis, prognosis and/or disease status of cancer, wherein a deviation or no deviation is indicative for the selection of the prophylactic or therapeutic treatment of the subject.

In still another aspect, an in vitro method for assessing the efficacy of a therapeutic treatment of cancer in a subject is provided, said method comprising determining the aggregate expression level of PTPs and the aggregate oxidation level of PTPs in a sample of the subject at a time point before the start of the treatment and one or more time points, such as regular intervals, after the start of the treatment, and comparing said aggregate expression level of PTPs and said aggregate oxidation level of PTPs in the first sample with said aggregate expression level of PTPs and said aggregate oxidation level of PTPs in one or more of the subsequent samples, wherein a deviation or no deviation is indicative for the efficacy of the therapeutic treatment in the subject.

In some embodiments, the aggregate expression level of all PTPs and the aggregate oxidation level of all PTPs present in the sample is determined.

In some embodiments, the aggregate expression level and the aggregate oxidation level of PTPs is determined in a group of PTPs comprising at least PTPN2, PTPRF, PTPRK, PTPN12, PTPN7, PTPN1, and PTPN6.

In some embodiments of the invention, the in vitro methods further comprise determining the expression level of one or more individual PTPs and/or the expression level of one or more individual oxidised PTPs and comparing said expression level(s) to a corresponding reference value or threshold value that is characteristic of a subject with a known diagnosis, prognosis and/or disease status of cancer, wherein a deviation or no deviation is indicative for the diagnosis, prognosis and/or disease status of the subject, or indicative for the selection of the prophylactic or therapeutic treatment of the subject, or indicative for the efficacy of the therapeutic treatment in the subject.

In another aspect, the use of the aggregate expression level of PTPs present in a sample in combination with the aggregate oxidation level of PTPs in the sample is provided for the diagnosis, prognosis and/or monitoring of cancer in a subject, or for determining the risk of developing cancer in a subject, or for assisting in the selection of a prophylactic or therapeutic treatment of cancer in a subject, or for assessing the efficacy of a therapeutic treatment of cancer. In certain embodiments, the use of the aggregate expression level of PTPs and the use of the aggregate oxidation level of PTPs is combined with the use of the expression level of one or more individual PTPs or oxidised PTPs, for the diagnosis, prognosis and/or monitoring of cancer in a subject, or for determining the risk of developing cancer in a subject, or for assisting in the selection of a prophylactic or therapeutic treatment of cancer in a subject, or for assessing the efficacy of a therapeutic treatment of cancer.

Other aspects of the application provide a kit for diagnosing, predicting, prognosing and/or monitoring cancer in a subject, for determining the risk of developing cancer in a subject, or for assisting in the selection of a prophylactic or therapeutic treatment of cancer in a subject, or for assessing the efficacy of a therapeutic treatment of cancer in a subject. Said kit comprises means for measuring the aggregate expression level of PTPs in a sample of the subject; means for measuring the aggregate oxidation level of PTPs in the sample of the subject; and a reference value or threshold value of the aggregate expression level and aggregate oxidation level of PTPs or means for establishing said reference value or threshold value, said reference value or threshold value represents a known diagnosis, prediction and/or prognosis of cancer, such as wherein said reference value or threshold value corresponds to the aggregate expression level of PTPs and to the aggregate oxidation level of PTPs in a sample not affected by cancer, such as in a healthy sample, or in a sample affected by cancer.

In some further aspects, the use of such a kit is provided for the diagnosis, prediction, prognosis and/or monitoring of cancer in a subject, or for determining the risk of developing cancer in a subject, or for assisting in the selection of a prophylactic or therapeutic treatment of cancer in a subject, or for assessing the efficacy of a therapeutic treatment of cancer in a subject.

### BRIEF DESCRIPTION OF DRAWINGS

FIG. 1. The crystal structure of human PTPN1 is shown with the active motif Cys-X₅-Arg. The pathway of protein tyrosine phosphatase inhibition by ROS. The conserved cysteine residue in the catalytic site of the PTP is critical for its activity.

FIG. 2. To determine total or aggregate PTP expression and oxidation, liver tissues will be homogenised in the presence DTT (DTT will reduce reversibly oxidised PTP), followed by pervanadate treatment (after buffer exchange; pervanadate will oxidise PTPs to the sulfonic state) and then digestion with trypsin. For oxidised PTPS NEM will be used to alkylate all reduced/active PTPs. Hyper-oxidised PTPs will be then detected by immune-precipitation (IP) and quantification will be achieved by mass spectrometry (MS).

FIG. 3. List and patient description of snap-frozen human liver samples for the initial PTP expression and oxidation profile analysis.

FIG. 4. PTP expression data from human samples. A. List and patient description of snap-frozen human liver samples for the initial and aggregate PTP expression and oxidation profile analysis. Human liver biopsies have been submitted to the indicated protocol to obtain the total list of expressed/oxidised PTP in the samples and a global protein profile. B. Lean, steatotic and NASH livers maintain a balanced PTP pool (aggregate PTP expression level). In HCC, the global PTP proteome (aggregate PTPs expression level) is reduced. In HCC, most PTPs are lost except for the core non-receptor PTPs (PTPN1, PTPN2 and PTPN6). C-E. Mass spectrometry data from the showed significant changes in individual PTP expression levels. PTPRF is one of the higher PTP in the steatotic cohort and its expression is lost in HCC.

### DESCRIPTION OF EMBODIMENTS

As used herein, the singular forms “a”, “an”, and “the” include both singular and plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. The terms also encompass “consisting of” and “consisting essentially of”, which enjoy well-established meanings in patent terminology.

The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of +/−10% or less, preferably +/−5% or less, more preferably +/−1% or less, and still more preferably +/−0.1% or less of and from the specified value, insofar such variations are appropriate to perform in the disclosed invention. It is to be understood that the value to which the modifier “about” refers is itself also specifically, and preferably, disclosed.

Whereas the terms “one or more” or “at least one”, such as one or more members or at least one member of a group of members, is clear per se, by means of further exemplification, the term encompasses inter alia a reference to any one of said members, or to any two or more of said members, such as, e.g., any ≥3, ≥4, ≥5, ≥6 or ≥7 etc. of said members, and up to all said members. In another example, “one or more” or “at least one” may refer to 1, 2, 3, 4, 5, 6, 7 or more.

The discussion of the background to the invention herein is included to explain the context of the invention. This is not to be taken as an admission that any of the material referred to was published, known, or part of the common general knowledge in any country as of the priority date of any of the claims.

Throughout this disclosure, various publications, patents and published patent specifications are referenced by an identifying citation. All documents cited in the present specification are hereby incorporated by reference in their entirety. In particular, the teachings or sections of such documents herein specifically referred to are incorporated by reference.

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the invention. When specific terms are defined in connection with a particular aspect of the invention or a particular embodiment of the invention, such connotation is meant to apply throughout this specification, i.e., also in the context of other aspects or embodiments of the invention, unless otherwise defined.

In the following passages, different aspects or embodiments of the invention are defined in more detail. Each aspect or embodiment so defined may be combined with any other aspect(s) or embodiment(s) unless clearly indicated to the contrary. In particular, any feature indicated as being preferred or advantageous may be combined with any other feature or features indicated as being preferred or advantageous.

Reference throughout this specification to “one embodiment”, “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention, and form different embodiments, as would be understood by those in the art. For example, in the appended claims, any of the claimed embodiments can be used in any combination.

With the present application, the inventors demonstrate that by evaluating the aggregate expression level and aggregate oxidation level of PTPs in tissue samples, they were able to identify samples with cancer, in particular with HCC, and more specifically obesity-induced HCC. More specifically, and as also further elaborated in the experimental part of this application, the inventors found that the global or total amount of PTPs in a sample, herein also referred to as the aggregate expression level of PTPs and represented in the PTP proteome, is reduced in HCC samples as compared to healthy tissue samples or samples derived from subjects with liver steatosis, NASH or NAFLD. In addition, they also found that the aggregate oxidation level of PTPs in a sample, which together with the aggregate PTP expression level forms a measure for their activity, also differs between healthy subjects and patients with HCC. The inventors thus present a novel method for the diagnosis of HCC by evaluating the aggregate expression level of PTPs in combination with the aggregate oxidation level of PTPs. Even further, the inventors found that the aggregate PTP activity, represented by the combination of the aggregate PTP expression level with the aggregate PTP oxidation profile, allows diagnosis of HCC. Surprisingly, the inventors found that most PTPs are lost in HCC samples, resulting in a considerable decrease of the aggregate expression of PTPs in HCC samples compared to samples from healthy controls, or patients with liver steatosis, or NASH. Hence the evaluation of the total or global amount of PTPs in combination with their oxidation profile in a tissue sample, and more specifically a liver tissue sample, is presented here as a novel biomarker for the diagnosis of HCC or for evaluating the disease progression of liver steatosis, NAFLD or NASH into HCC. Hence, the present methods may evaluate the overall or collective amount or expression level of PTPs, wherein a reduction in this amount serves as an indication of HCC or progression to HCC, and the present methods additionally determine the fraction of the global PTPs that are oxidised (regardless of whether the global PTP amount is reduced or not), wherein increased PTP oxidation serves as an indication of HCC or progression to HCC. In certain embodiments, the total PTP activity or capacity may be estimated based on the overall and aggregate expression level of PTPs in the sample, reduced with the fraction of PTPs that are oxidised, such that the total PTP activity or capacity in a sample corresponds to those PTPs present in the sample that are not oxidised (and hence deemed active).

Furthermore, by evaluation of the differential expression of the different individual PTPs, the inventors could differentially diagnose between liver steatosis, NAFLD, NASH or HCC. The inventors found that there are significant changes in the expression of individual PTPs during the different stages of liver steatosis, NAFLD, NASH and HCC. This change in expression will constitute changes in the capacity of the total or overall PTP activity.

Accordingly, in some aspects, the application provides an in vitro method for the diagnosis, prognosis and/or monitoring of cancer in a subject, for determining the risk of developing cancer in a subject, for assisting in the selection of a prophylactic or therapeutic treatment of cancer in a subject, or for assessing the efficacy of a therapeutic treatment of cancer in a subject. Said methods are typically characterized in that the aggregate expression level of PTPs and the aggregate oxidation level of PTPs in a sample of the subject is determined, and that said aggregate expression level and aggregate oxidation level of PTPs is compared to a corresponding reference value or threshold value that is characteristic of a subject with a known diagnosis, prognosis and/or disease status of cancer in the subject. Hence, in the in vitro method for the diagnosis, prognosis and/or monitoring of cancer in a subject, a deviation or no deviation of the aggregate expression level and oxidation level of PTPs of the sample as compared to the corresponding reference value or threshold value is indicative for the diagnosis, prognosis and/or disease status of cancer in the subject. In the in vitro method for determining the risk of developing cancer, a deviation or no deviation of the aggregate expression level and aggregate oxidation level of PTPs in the sample as compared to a corresponding reference value or threshold value is indicative for the risk of developing cancer in the subject. In the in vitro method for assisting in the selection of a prophylactic or therapeutic treatment for cancer in the subject, a deviation or no deviation of the aggregate expression level and aggregate oxidation level of PTPs of the sample as compared to the corresponding reference value or threshold value is indicative for the selection of the prophylactic or therapeutic treatment of the subject. And in the in vitro method for assessing the efficacy of a therapeutic treatment of cancer in a subject, the aggregate expression level and the aggregate oxidation level of PTPs is determined at a time point before the start of the treatment and one or more time points after the start of the treatment. Said aggregate expression level and oxidation level of PTPs is then compared with the aggregate expression level and oxidation level of PTPs in one or more subsequent samples, wherein a deviation or no deviation is indicative for the efficacy of the therapeutic treatment in the subject.

The inventors of the present application thus found that the aggregate expression level of PTPs present in a sample in combination with the aggregate oxidation level of PTPs present in a sample can be used as a biomarker in cancer, in particular in HCC. In the context of this application, the aggregate expression level of PTPs is to be understood as the total or global amount of PTPs that is measurable in a sample or that can be determined or calculated based on measurements performed on a sample. The aggregate oxidation level of PTPs is to be understood as the total or global oxidation level of PTPs that is measurable in a sample or that can be determined or calculated based on measurements performed on a sample. In some situations, an observed decrease in the overall or aggregate expression level or amount of PTPs present in a sample will already allow for the conclusion that the aggregate activity of PTPs in that sample is decreased. In other preferred situations, regardless of whether the global PTP level in a sample is decreased or not, one may wish to also evaluate the degree of oxidation of PTPs in the sample, i.e., determine the fraction of the collective PTPs in the sample that have been oxidised at their active site cysteine and thus rendered inactive, or that are not oxidised and hence presumed active. Such oxidation measurements may then feed into the overall determination of the aggregate PTPs activity in the sample. Hence, the in vitro methods of the present invention combine determining or measuring the aggregate expression level of PTPs in a sample with determining or measuring the aggregate oxidation level of PTPs in the sample. In said methods, the aggregate expression level of PTPs is thus combined with the aggregate oxidation level of PTPs, thereby representing the aggregate or global activity of PTPs in a sample. In the context of this application, the aggregate activity of PTPs is thus reflected by the general, global or aggregate expression level of PTPs in combination with the oxidation level of the PTPs. More specifically, the aggregate activity of PTPs is reflected by the overall and aggregate expression level of PTPs, reduced with the fraction of the PTPs that are oxidised, such that the total PTP activity or capacity in a sample corresponds to those PTPs present in the sample that are not oxidised (and hence deemed active). As further detailed below and also shown in the examples, the global or aggregate expression level of the PTPs and the global or aggregate oxidation level of PTPs in a sample is thus determined on a general or global level, without determining the expression or oxidation levels of the individual PTPs in the sample separately and on an individual basis.

In some embodiments, the aggregate expression level of all PTPs and the aggregate oxidation level of all PTPs present in the sample is determined and represents the aggregate or global activity of PTPs in the sample.

In some embodiments, the aggregate expression level and the aggregate oxidation level of PTPs is determined in a group of PTPs comprising at least PTPN2, PTPRF, PTPRK, PTPN12, PTPN7, PTPN1, and PTPN6. In said embodiments, the aggregate expression level and the aggregate oxidation level of PTPs determined in a group of PTPs comprising at least PTPN2, PTPRF, PTPRK, PTPN12, PTPN7, PTPN1, and PTPN6 represents the aggregate or global activity of PTPs in the sample.

In some embodiments, the aggregate expression level and the aggregate oxidation level of PTPs is determined in a group of PTPs comprising at least PTPRB, PTPRD, PTPRF, PTPRM, PTPRG, PTPRK, PTPRJ, PTPRC, PTPRA, PTPRE, PTPN1, PTPN2, PTPN3, PTPN6, PTPN7, PTPN9, PTPN11 and PTPN12. In said embodiments, the aggregate expression level and the aggregate oxidation level of PTPs determined in a group of PTPs comprising at least PTPRB, PTPRD, PTPRF, PTPRM, PTPRG, PTPRK, PTPRJ, PTPRC, PTPRA, PTPRE, PTPN1, PTPN2, PTPN3, PTPN6, PTPN7, PTPN9, PTPN11 and PTPN12 represents the aggregate or global activity of PTPs in the sample.

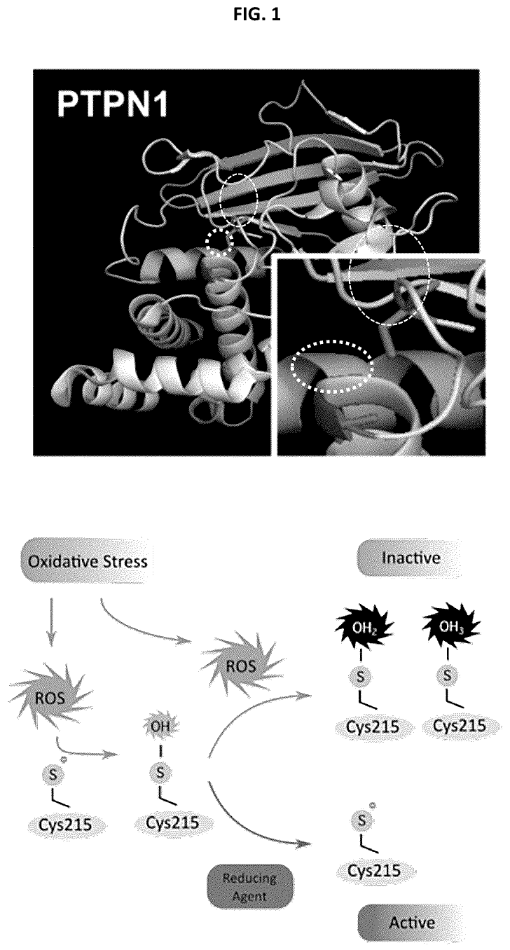

PTPs were initially described as a small family of ubiquitously expressed enzymes with generalized housekeeping roles. They can be subdivided into 21 different receptor-like members, and 17 non-transmembrane members and studies have shown that PTPs are inactivated by ROS (also see FIG. 1). Some of the PTPs can occur in different isoforms. Further, some PTPs can have two different catalytic sites, such as for example PTPRF, represented as PTPRF-1 and PTPRF-2, PTPRA, represented as PTPRA-1 and PTPRA-2, and PTPRE, represented as PTPRE-1 and PTPRE-2. Previously, the levels of PTPs were generally determined based on the levels of each of the individual PTPs separately. As shown in the experimental part of this application, the inventors now developed a mass spectrometry method that quantitatively measures the global or aggregate PTP protein expression profile in a tissue sample, more specific a liver tissue sample, without measurement of each of the PTPs individually. With this method, the inventors found that the global protein expression level of the PTPs in general, as evaluated by mass spectrometry, was markedly reduced in HCC tumour samples as compared to healthy liver tissue samples or liver tissue samples from subjects with liver steatosis, NAFLD, or NASH. Furthermore, the inventors also found that global or aggregate oxidation of the PTPs corresponds to the disease status in HCC. Hence, from a methodology standpoint, reference to aggregate, cumulative, global, total or overall activity, expression level, amount, or oxidation status of PTPs may refer to the respective measurements done on the PTP group of enzymes as a whole. This does not exclude that the measurement methodology may also be able to discriminate between the different PTPs present in the sample, insofar it can provide a readout on the PTPs all together. By means of an example and without limitation, methodologies that take advantage of the presence of the highly conserved active site in PTPs to separate/isolate and subsequently quantify substantially all PTPs from a sample in unison, may be particularly suitable for the purposes of the present methods.

The in vitro methods according to the different aspects of the invention are thus characterized by determining the aggregate expression level and aggregate oxidation level of PTPs in a sample and comparing said level to a corresponding reference value or threshold value. Thus, the aggregate activity of PTPs is determined and represented by the aggregate expression level of PTPs in combination with the aggregate oxidation level of PTPs in a sample. The aggregate expression level of PTPs and the aggregate oxidation level of PTPs can thus be determined on a general level without measurement of their individual levels separately. As such, in some embodiments, the PTPs are selected from all PTPs available in the sample. Thus, in some embodiments, the aggregate expression level of all PTPs and the aggregate oxidation level of all PTPs present in the sample is determined. In some embodiments, the aggregate expression level and the aggregate oxidation level of PTPs is determined in a group of PTPs comprising at least PTPN2, PTPRF, PTPRK, PTPN12, PTPN7, PTPN1, and PTPN6. In some other embodiments, the aggregate expression level and the aggregate oxidation level of PTPs is determined in a group of PTPs comprising at least PTPRB, PTPRD, PTPRF, PTPRM, PTPRG, PTPRK, PTPRJ, PTPRC, PTPRA, PTPRE, PTPN1, PTPN2, PTPN3, PTPN6, PTPN7, PTPN9, PTPN11 and PTPN12. For example, when determining the aggregate expression level of PTPs, the expression level can be determined in a global manner, thereby measuring the expression level of all PTPs in a general way without specifically determining the expression level of each PTP individually and without making the sum of these individual expression levels. In some embodiments, the aggregate expression level of PTPs is determined in a group of PTPs comprising at least PTPN2, PTPRF, PTPRK, PTPN12, PTPN7, PTPN1, and PTPN6; or in a group comprising at least PTPRB, PTPRD, PTPRF, PTPRM, PTPRG, PTPRK, PTPRJ, PTPRC, PTPRA, PTPRE, PTPN1, PTPN2, PTPN3, PTPN6, PTPN7, PTPN9, PTPN11 and PTPN12, wherein the PTP expression level in this group is determined in a global manner without specifically determining the expression level of each PTP individually and without making the sum of these individual expression levels. This can for example be done by using a universal antibody that detects the catalytic site of all PTPs.

In some other embodiments, the aggregate expression level of PTPs can be determined on an individual basis by individually determining the expression of each PTP separately followed by calculating their total amount based on these individual levels. In an embodiment, the PTPs can be selected from PTPN2, PTPRF, PTPRK, PTPN12, PTPN7, PTPN1, and PTPN6, wherein the aggregate expression level of PTPs is determined by making the sum of the individual expression level of at least each of these PTPs. In a related embodiment, the PTPs can be selected from PTPRB, PTPRD, PTPRF, PTPRM, PTPRG, PTPRK, PTPRJ, PTPRC, PTPRA, PTPRE, PTPN1, PTPN2, PTPN3, PTPN6, PTPN7, PTPN9, PTPN11 and/or PTPN12. In still another embodiment, the PTPs comprise at least PTPRF, PTPRK, PTPN1, PTPN2, PTPN6, PTPN7, and PTPN12, wherein the aggregate expression level of PTPs is determined by making the sum of the individual expression levels of at least each of these PTPs.

Similarly, when determining the aggregate oxidation level of PTPs, this can be determined on a global level without determining the level of each oxidised PTP individually. In some embodiments, the aggregate oxidation level of PTPs is determined in a group of PTPs comprising at least PTPN2, PTPRF, PTPRK, PTPN12, PTPN7, PTPN1, and PTPN6, wherein the PTP oxidation level in this group is determined in a global manner without specifically determining the oxidation level of each PTP individually and without making the sum of these individual oxidation levels. In some embodiments, the aggregate oxidation level of PTPs is determined in a group of PTPs comprising at least PTPRB, PTPRD, PTPRF, PTPRM, PTPRG, PTPRK, PTPRJ, PTPRC, PTPRA, PTPRE, PTPN1, PTPN2, PTPN3, PTPN6, PTPN7, PTPN9, PTPN11 and PTPN12, wherein the PTP oxidation level in this group is determined in a global manner without specifically determining the oxidation level of each PTP individually and without making the sum of these individual oxidation levels.

In some embodiments, the aggregate oxidation level of PTPs can be determined on an individual basis by individually determining the oxidation level of each PTP separately followed by calculating their total amount based on these individual levels. In an embodiments, the PTPs can be selected from PTPN2, PTPRF, PTPRK, PTPN12, PTPN7, PTPN1, and PTPN6. In a related embodiment, the PTPs can be selected from PTPRB, PTPRD, PTPRF, PTPRM, PTPRG, PTPRK, PTPRJ, PTPRC, PTPRA, PTPRE, PTPN1, PTPN2, PTPN3, PTPN6, PTPN7, PTPN9, PTPN11 and/or PTPN12.

In still another embodiment, the aggregate expression level of PTPs and/or the aggregate oxidation level of PTPs is determined by evaluating at least 2; preferably at least 5 PTPs and/or oxidised PTPs selected from PTPRB, PTPRD, PTPRF, PTPRM, PTPRG, PTPRK, PTPRJ, PTPRC, PTPRA, PTPRE, PTPN1, PTPN2, PTPN3, PTPN6, PTPN7, PTPN9, PTPN11 and/or PTPN12. In still a related embodiment, the aggregate expression level and/or oxidation level of PTPs is determined by evaluating at least the expression levels of PTPRF, PTPRK, PTPN1, PTPN2, PTPN6, PTPN7, and PTPN12, followed by calculating their total amount based on these individual expression levels in combination with their oxidised forms. In still some other embodiments, the global or aggregate expression level and/or oxidation level of PTPs as taught herein is combined with the individual expression level of oxidised PTPRF, PTPRK, PTPN1, PTPN2, PTPN6, PTPN7, or PTPN12; in particular with the individual expression level of oxidized PTPN1, oxidized PTPN2 and oxidised PTPN6.

In some embodiments, the in vitro methods of the invention may further comprise determining the expression level of one or more individual PTPs and/or the expression level of one or more oxidized PTPs, and comparing said expression level of one or more individual PTPs and/or one or more individual oxidised PTPs to a corresponding reference value or threshold value that is characteristic of a subject with a known diagnosis, prognosis and/or disease status of the subject, or indicative for the risk of developing cancer, or indicative for the selection of the prophylactic or therapeutic treatment of the subject, or indicative for the efficacy of the therapeutic treatment in the subject. In said context, the global or aggregate expression level of PTPs and the aggregate oxidation level of PTPs as taught herein, is then combined with the expression level of one or more of the individual PTPs and/or individual oxidized PTPs. In a further embodiment, the individual PTPs are selected from PTPRB, PTPRD, PTPRF, PTPRM, PTPRG, PTPRK, PTPRJ, PTPRC, PTPRA, PTPRE, PTPN1, PTPN2, PTPN3, PTPN6, PTPN7, PTPN9, PTPN11 and/or PTPN12. In still a further embodiment, the expression level of at least 2, preferably at least 5, of the individual PTPs and/or individual oxidized PTPs selected from PTPRB, PTPRD, PTPRF, PTPRM, PTPRG, PTPRK, PTPRJ, PTPRC, PTPRA, PTPRE, PTPN1, PTPN2, PTPN3, PTPN6, PTPN7, PTPN9, PTPN11 and/or PTPN12 are determined. In still other embodiments, the global or aggregate expression level of PTPs and the aggregate oxidation level of PTPs, is combined with the individual expression level PTPRF, PTPRK, PTPN1, PTPN2, PTPN6, PTPN7, and PTPN12, whether or not in combination with their oxidised forms. In still some other embodiments, the global or aggregate expression level of PTPs and the aggregate oxidation level of PTPs, is combined with the individual expression level of oxidised PTPRF, PTPRK, PTPN1, PTPN2, PTPN6, PTPN7, or PTPN12; in particular with the individual expression level of oxidized PTPN1, oxidized PTPN2 and oxidised PTPN6.

Thus, in some aspects, in vitro methods are provided for the diagnosis, prognosis and/or monitoring of cancer in a subject, said methods comprising determining the aggregate expression level of PTPs in a sample of the subject, determining the aggregate oxidation level of PTPs in the sample, optionally determining the expression level of one or more individual PTPs and/or oxidized PTPs, and comparing the aggregate expression level of PTPs and the aggregate oxidation level of PTPs, and optionally the expression level of one or more individual PTPs and/or oxidized PTPs to a corresponding reference value or threshold value of said PTPs that is characteristic of a subject with a known diagnosis, prognosis and/or disease status of cancer, wherein a deviation or no deviation is indicative for the diagnosis, prognosis and/or disease status of cancer in the subject. In some embodiments, the aggregate expression level of all PTPs and the aggregate oxidation level of all PTPs present in the sample is determined and represents the aggregate or global activity of PTPs in the sample. In some embodiments, the aggregate expression level and the aggregate oxidation level of PTPs is determined in a group of PTPs comprising at least PTPN2, PTPRF, PTPRK, PTPN12, PTPN7, PTPN1, and PTPN6. In some embodiments, the aggregate expression level and the aggregate oxidation level of PTPs is determined in a group of PTPs comprising at least PTPRB, PTPRD, PTPRF, PTPRM, PTPRG, PTPRK, PTPRJ, PTPRC, PTPRA, PTPRE, PTPN1, PTPN2, PTPN3, PTPN6, PTPN7, PTPN9, PTPN11 and PTPN12. In some further embodiments, the individual PTPs and/or individual oxidised PTPs are selected from PTPRB, PTPRD, PTPRF, PTPRM, PTPRG, PTPRK, PTPRJ, PTPRC, PTPRA, PTPRE, PTPN1, PTPN2, PTPN3, PTPN6, PTPN7, PTPN9, PTPN11 and/or PTPN12. In a preferred embodiment, at least 2, preferably at least 5, of said individual PTPs and/or individual oxidised PTPs are determined. In still some other preferred embodiments, the aggregate expression level and/or oxidation level of PTPs is determined in combination with the individual expression level of PTPRF, PTPRK, PTPN1, PTPN2, PTPN6, PTPN7, and/or PTPN12; even further, in combination with the individual expression level of oxidized PTPN1, oxidized PTPN2 and oxidised PTPN6.

In some other aspects, in vitro methods are provided for determining the risk of developing cancer in a subject, said methods comprising determining the aggregate expression level of PTPs in a sample of the subject, determining the aggregate oxidation level of PTPs in the sample, optionally determining the expression level of one or more individual PTPs and/or oxidized PTPs, and comparing the aggregate expression level of PTPs in the sample and the aggregate oxidation level of PTPs in the sample, and optionally the expression level of one or more individual PTPs and/or oxidized PTPs to a corresponding reference value or threshold value of said PTPs that is characteristic of a subject with a known diagnosis, prognosis and/or disease status of cancer, wherein a deviation or no deviation is indicative for the risk of developing cancer in the subject. In some embodiments, the aggregate expression level of all PTPs and the aggregate oxidation level of all PTPs present in the sample is determined and represents the aggregate or global activity of PTPs in the sample. In some embodiments, the aggregate expression level and the aggregate oxidation level of PTPs is determined in a group of PTPs comprising at least PTPN2, PTPRF, PTPRK, PTPN12, PTPN7, PTPN1, and PTPN6. In some embodiments, the aggregate expression level and the aggregate oxidation level of PTPs is determined in a group of PTPs comprising at least PTPRB, PTPRD, PTPRF, PTPRM, PTPRG, PTPRK, PTPRJ, PTPRC, PTPRA, PTPRE, PTPN1, PTPN2, PTPN3, PTPN6, PTPN7, PTPN9, PTPN11 and PTPN12. In some further embodiments, the individual PTPs and/or individual oxidised PTPs are selected from PTPRB, PTPRD, PTPRF, PTPRM, PTPRG, PTPRK, PTPRJ, PTPRC, PTPRA, PTPRE, PTPN1, PTPN2, PTPN3, PTPN6, PTPN7, PTPN9, PTPN11 and/or PTPN12. In a preferred embodiment, at least 2, preferably at least 5, of said individual PTPs and/or individual oxidised PTPs are determined. In still some other preferred embodiments, the aggregate expression level and oxidation level of PTPs is determined in combination with the individual expression level of PTPRF, PTPRK, PTPN1, PTPN2, PTPN6, PTPN7, and/or PTPN12; even further, in combination with the individual expression level of oxidized PTPN1, oxidized PTPN2 and oxidised PTPN6.

In still some other aspects, in vitro methods are provided for the selection of a prophylactic or therapeutic treatment of cancer in a subject, said methods comprising determining the aggregate expression level of PTPs in a sample of the subject, determining the aggregate oxidation level of PTPs in a sample, optionally determining the expression level of one or more individual PTPs and/or oxidized PTPs, and comparing the aggregate expression of PTPs and the aggregate oxidation level of PTPs, and optionally the expression level of one or more individual PTPs and/or oxidized PTPs to a corresponding reference value or threshold value of said PTPs that is characteristic of a subject with a known diagnosis, prognosis and/or disease status of cancer, wherein a deviation or no deviation is indicative for the selection of the prophylactic or therapeutic treatment of the subject. In some embodiments, the aggregate expression level of all PTPs and the aggregate oxidation level of all PTPs present in the sample is determined and represents the aggregate or global activity of PTPs in the sample. In some embodiments, the aggregate expression level and the aggregate oxidation level of PTPs is determined in a group of PTPs comprising at least PTPN2, PTPRF, PTPRK, PTPN12, PTPN7, PTPN1, and PTPN6. In some embodiments, the aggregate expression level and the aggregate oxidation level of PTPs is determined in a group of PTPs comprising at least PTPRB, PTPRD, PTPRF, PTPRM, PTPRG, PTPRK, PTPRJ, PTPRC, PTPRA, PTPRE, PTPN1, PTPN2, PTPN3, PTPN6, PTPN7, PTPN9, PTPN11 and PTPN12. In some further embodiments, the individual PTPs and/or individual oxidised PTPs are selected from PTPRB, PTPRD, PTPRF, PTPRM, PTPRG, PTPRK, PTPRJ, PTPRC, PTPRA, PTPRE, PTPN1, PTPN2, PTPN3, PTPN6, PTPN7, PTPN9, PTPN11 and/or PTPN12. In a preferred embodiment, at least 2, preferably at least 5, of said individual PTPs and/or individual oxidised PTPs are determined. In still some other preferred embodiments, the aggregate expression level and/or oxidation level of PTPs is determined in combination with the individual expression level of PTPRF, PTPRK, PTPN1, PTPN2, PTPN6, PTPN7, and/or PTPN12; even further, in combination with the individual expression level of oxidized PTPN1, oxidized PTPN2 and oxidised PTPN6.

In some other aspects, in vitro methods are provided for assessing the efficacy of a therapeutic treatment of cancer in a subject, said methods comprising determining the aggregate expression level of PTPs in a sample of the subject at a time point before the start of the treatment and one or more time points, such as at regular intervals, after the start of the treatment, determining the aggregate oxidation level of PTPs in the sample at a time point before the start of the treatment and one or more time points, such as at regular intervals, after the start of the treatment, optionally determining the expression level of one or more individual PTPs and/or oxidized PTPs at a time point before the start of the treatment and one or more time points, such as at regular intervals, after the start of the treatment, and comparing the aggregate expression and oxidation level of PTPs and optionally the expression level of one or more individual PTPs and/or oxidized PTPs in the first sample with that in one or more of the subsequent samples, wherein a deviation or no deviation is indicative for the efficacy of the therapeutic treatment in the subject. In some embodiments, the aggregate expression level of all PTPs and the aggregate oxidation level of all PTPs present in the sample is determined and represents the aggregate or global activity of PTPs in the sample. In some embodiments, the aggregate expression level and the aggregate oxidation level of PTPs is determined in a group of PTPs comprising at least PTPN2, PTPRF, PTPRK, PTPN12, PTPN7, PTPN1, and PTPN6. In some embodiments, the aggregate expression level and the aggregate oxidation level of PTPs is determined in a group of PTPs comprising at least PTPRB, PTPRD, PTPRF, PTPRM, PTPRG, PTPRK, PTPRJ, PTPRC, PTPRA, PTPRE, PTPN1, PTPN2, PTPN3, PTPN6, PTPN7, PTPN9, PTPN11 and PTPN12. In some further embodiments, the individual PTPs and/or individual oxidised PTPs are selected from PTPRB, PTPRD, PTPRF, PTPRM, PTPRG, PTPRK, PTPRJ, PTPRC, PTPRA, PTPRE, PTPN1, PTPN2, PTPN3, PTPN6, PTPN7, PTPN9, PTPN11 and/or PTPN12. In a preferred embodiment, at least 2, preferably at least 5, of said individual PTPs and/or individual oxidised PTPs are determined. In still some other preferred embodiments, the aggregate expression level and oxidation level of PTPs is determined in combination with the individual expression level of PTPRF, PTPRK, PTPN1, PTPN2, PTPN6, PTPN7, and/or PTPN12; even further, in combination with the individual expression level of oxidized PTPN1, oxidized PTPN2 and oxidised PTPN6.

In the context of the present application, “diagnosis” and “diagnosing” generally include a determination of a subject's susceptibility to a disease or disorder, a determination as to whether a subject is presently affected by a disease or disorder, a prognosis of a subject affected by a disease or disorder, and therametrics (e.g. monitoring a subject's condition to provide information as to the effect or efficacy of therapy).

The terms “prognosis” or “prognose” refer to the act or art of foretelling the course of a disease. Additionally, the terms refer to the prospect of survival recovery from a disease as anticipated from the usual course of that disease or indicated by special features of the individual case. Further, the terms refer to the art or act of identifying a disease from its signs and symptoms.

In the present application, the aggregate expression level of PTPs, the aggregate oxidation level of PTPs, and/or the individual expression level of PTPs selected from PTPRB, PTPRD, PTPRF, PTPRM, PTPRG, PTPRK, PTPRJ, PTPRC, PTPRA, PTPRE, PTPN1, PTPN2, PTPN3, PTPN6, PTPN7, PTPN9, PTPN11 and/or PTPN12 are considered as biomarkers for cancer, more specifically for hepatocellular cancer (HCC). In some aspects, said biomarkers can also be used to differentiate between the different disease states such as liver steatosis, NAFLD, NASH and/or HCC. As used herein, a “biomarker” is widespread in the art and may broadly denote a biological molecule and/or detectable portion thereof whose qualitative and/or quantitative evaluation in a subject is predictive or informative (e.g., predictive, diagnostic and/or prognostic) with respect to one or more aspects of the subject's phenotype and/or genotype, such as, for example, with respect to the status of the subject as to a given disease or condition. Reference is made herein to a “biomarker panel” if more than one biomarker is being detected in the methods or uses as taught herein.

In certain embodiments, a biomarker as taught herein, may be peptide-, polypeptide- and/or protein-based.

The reference to any marker, including any peptide, polypeptide, protein, corresponds to the marker, peptide, polypeptide, protein, commonly known under the respective designations in the art. The terms encompass such markers, peptides, polypeptides, proteins of any organism where found, and particularly of animals, preferably warm-blooded animals, more preferably vertebrates, yet more preferably mammals, including humans and non-human mammals, still more preferably of humans. The terms particularly encompass such markers, peptides, polypeptides, proteins with a native sequence, i.e., ones of which the primary sequence is the same as that of the markers, peptides, polypeptides, proteins found in or derived from nature. A skilled person understands that native sequences may differ between different species due to genetic divergence between such species. Moreover, native sequences may differ between or within different individuals of the same species due to normal genetic diversity (variation) within a given species. Also, native sequences may differ between or even within different individuals of the same species due to post-transcriptional or posttranslational modifications. Any such variants or isoforms of markers, peptides, polypeptides, proteins are intended herein. Accordingly, all sequences of markers, peptides, polypeptides, or proteins found in or derived from nature are considered “native”. The terms encompass the markers, peptides, polypeptides, or proteins when forming a part of a living organism, organ, tissue or cell, when forming a part of a biological sample, as well as when at least partly isolated from such sources. The terms also encompass markers, peptides, polypeptides, or proteins when produced by recombinant or synthetic means.

In certain embodiments, the biomarkers as taught herein, may be a human biomarker, such as in particular the aggregate activity of PTPs, the aggregate expression level of PTPs, the aggregate oxidation level of PTPs, or the individual human proteins selected from the group consisting of PTPRB, PTPRD, PTPRF, PTPRM, PTPRG, PTPRK, PTPRJ, PTPRC, PTPRA, PTPRE, PTPN1, PTPN2, PTPN3, PTPN6, PTPN7, PTPN9, PTPN11 and/or PTPN12, and/or their oxidised form. In preferred embodiments, the biomarkers are further selected from PTPRF, PTPN1, PTPRK, PTPN2, PTPN6, PTPN7, and/or PTPN12 and/or their oxidised forms.

Unless otherwise apparent from the context, reference herein to any marker, peptide, polypeptide or protein, or fragment thereof may generally also encompass modified forms of said marker, peptide, polypeptide, or protein, or fragment thereof, such as bearing post-expression modifications including, for example, phosphorylation, glycosylation, lipidation, methylation, cysteinylation, sulphonation, glutathionylation, acetylation, oxidation, and the like.

The reference herein to any marker, peptide, polypeptide or protein also encompasses fragments thereof. Hence, the reference herein to measuring (or measuring the quantity of), determining the presence, or determining the expression level of any one marker, peptide, polypeptide or protein may encompass measuring, determining the presence or determining the expression level of the marker, peptide, polypeptide, or protein, such as, e.g. measuring any mature and/or processed soluble/secreted form(s) thereof (e.g., plasma circulating form(s)) and/or measuring one or more fragments thereof.

For example, any marker, peptide, polypeptide or protein, and/or one or more fragments thereof may be measured collectively, such that the measured quantity corresponds to the sum amounts of the collectively measured species. In a further example, any marker, peptide, polypeptide or protein and/or one or more fragments thereof may be measured each individually.

The term “fragment” with reference to a peptide, polypeptide, or protein generally denotes a N- and/or C-terminally truncated form of the peptide, polypeptide, or protein. Preferably, a fragment may comprise at least about 30%, e.g., at least about 50% or at least about 70%, preferably at least about 80%, e.g., at least about 85%, more preferably at least about 90%, and yet more preferably at least about 95% or even about 99% of the amino acid sequence length of said peptide, polypeptide, or 10 protein. For example, insofar not exceeding the length of the full-length peptide, polypeptide, or protein, a fragment may include a sequence of ≥5 consecutive amino acids, or ≥10 consecutive amino acids, or ≥20 consecutive amino acids, or ≥30 consecutive amino acids, e.g., ≥40 consecutive amino acids, such as for example ≥50 consecutive amino acids, e.g., ≥60, ≥70, ≥80, ≥90, ≥100, ≥200, ≥300 or ≥400 consecutive amino acids of the corresponding full-length peptide, polypeptide, or protein.

The reference herein to any protein, polypeptide or peptide may also encompass variants thereof. The term “variant” of a protein, polypeptide or peptide refers to proteins, polypeptides or peptides the sequence (i.e. amino acid sequence) of which is substantially identical (i.e. largely but not wholly identical) to the sequence of said recited protein or polypeptide, e.g. at least about 80% identical or at least about 85% identical, e.g. preferably at least about 90% identical, e.g., at least 91% identical, 92% identical, more preferably at least about 93% identical, e.g. at least 94% identical, even more preferably at least about 95% identical, e.g., at least 96% identical, yet more preferably at least about 97% identical, e.g. at least 98% identical, and most preferably at least 99% identical. Preferably, a variant may display such degrees of identity to a recited protein, polypeptide or peptide when the whole sequence of the recited protein, polypeptide or peptide is queried in the sequence alignment (i.e, overall sequence identity).

A variant of a protein, polypeptide or peptide may be a homologue (e.g, orthologue or paralogue) of said protein, polypeptide or peptide. As used herein, the term “homology” generally denotes structural similarities between macromolecules, particularly between two proteins or polypeptides, from same or different taxons, wherein said similarity is due to shared ancestry.

Where the present specification refers to or encompasses fragments and/or variants of proteins, polypeptides or peptides, this preferably denotes variants and/or fragments which are “functional”, i.e., which at least partly retain the biological activity or intended functionality of the respective proteins, polypeptides or peptides. Preferably, a functional fragment and/or variant may retain at least about 20%. e.g., at least 30%, or at least about 40%, or at least about 50%, e.g., at least 60%, more preferably at least about 70%, e.g., at least 80%, yet more preferably at least about 85%, still more preferably at least about 90%, and most preferably at least about 95% or even about 100% or higher of the intended biological activity or functionality compared to the corresponding protein, polypeptide or peptide.

In some embodiments, the present invention thus allows to diagnose, prognose, or monitor cancer. In some further embodiments, the cancer can be selected from hepatocellular carcinoma (HCC), breast cancer, pancreatic cancer, lung cancer, colon cancer, prostate cancer, melanoma, multiple myeloma, lymphoma, and/or leukemia. Preferably the cancer is HCC; even more preferably wherein the cancer is obesity-induced HCC.

As evidenced from the experimental part, the aggregate expression level of PTPs in combination with the aggregate oxidation level of PTPs allows to differentiate between HCC and healthy subjects or subjects with liver steatosis, NAFLD or NASH. The methods of the present invention thus allow to differentiate between healthy subjects or subjects with a non-malignant liver disease and HCC. The methods of the invention are therefore useful to identify when a non-malignant liver disease might evaluate into a malignant HCC, thereby identifying the malignant HCC at an early stage.

As used in the context of the present application, liver steatosis or hepatic steatosis is understood as a wide range of liver disorders characterized by an excessive accumulation of fat (fatty acids and triglycerides) in liver cells (hepatocytes). Liver steatosis is also referred to as fatty liver, and subjects with liver steatosis do not always show a disturbed liver function yet. Early stage of liver steatosis that occurs in subject who do not drink alcohol or who drink very moderately is called non-alcoholic fatty liver disease (NAFLD). NAFLD is one of the causes of fatty liver, occurring when fat is deposited in the liver due to causes other than excessive alcohol use. It is the most common liver disorder in developed countries and it is generally associated with factors of the metabolic syndrome. Non-alcoholic steatohepatitis (NASH) is a disease state in which liver steatosis is combined with inflammation and fibrosis (steatohepatitis), resulting in a disturbed liver function. NASH is regarded as a major cause of cirrhosis of the liver of unknown cause. Liver steatosis, NAFLD and NASH are known to progress to hepatocellular carcinoma (HCC) or liver cancer. As used herein, hepatocellular carcinoma (HCC) refers to a malignant tumour occurring in the liver. In some embodiments, the HCC is further specified as obesity-induced HCC. The term “obesity” is to be understood with the usual meaning in the medical field as a chronic disease of preventable multifactorial origin characterized by excessive accumulation of fat or general hypertrophy of adipose tissue in the body; that is, when the natural energy reserve of humans and other animals, stored in the form of body fat is increased to a point where it is associated with numerous complications such as certain health conditions or diseases and increase in mortality.

As indicated herein, the methods according to some embodiments of this application, may comprise determining the presence, expression level and/or oxidation level of at least one of the proteins selected from the group consisting of PTPRB, PTPRD, PTPRF, PTPRM, PTPRG, PTPRK, PTPRJ, PTPRC, PTPRA, PTPRE, PTPN1, PTPN2, PTPN3, PTPN6, PTPN7, PTPN9, PTPN11 and PTPN12 in a sample from the subject. In a further embodiment, at least two, at least three at least four, at least five, at least six, or all proteins are selected from the group consisting of PTPRB, PTPRD, PTPRF, PTPRM, PTPRG, PTPRK, PTPRJ, PTPRC, PTPRA, PTPRE, PTPN1, PTPN2, PTPN3, PTPN6, PTPN7, PTPN9, PTPN11 and/or PTPN12. In still some further embodiments, the selected PTPs comprise at least PTPRF, PTPRK, PTPN1, PTPN2, PTPN6, PTPN7, and PTPN12.

In another aspect of the invention, and as disclosed herein, in vitro methods for assisting in the selection of a prophylactic or therapeutic treatment of HCC in a subjects are disclosed. Also in vitro methods for assessing the efficacy of a therapeutic treatment of HCC in a subject are provided.

The terms “treatment”, “treating”, “treat” and the like refer to obtaining a desired pharmacological and/or physiological effect. The effect may be prophylactic in terms of completely or partially preventing a disease or symptom thereof and/or may be therapeutic in terms of a partial or complete stabilization or cure for a disease and/or adverse effect attributable to the disease. “Treatment” covers any treatment of a disease in a mammal, particular a human, and includes: (a) preventing the disease or symptom but has not yet been diagnosed as having it; (b) inhibiting the disease symptoms, i.e. arresting its development: or (c) relieving the disease symptoms, i.e. causing regression of the disease or symptom. Beneficial or desired clinical results may include, without limitation, alleviation of one or more symptoms or one or more biological markers, diminishment of extent of disease, stabilised (i.e., not worsening) state of disease, delay or slowing of disease progression, amelioration or palliation of the disease state, and the like. “Treatment” can also mean prolonging survival as compared to expected survival if not receiving treatment. Non-limiting example of therapeutic treatment of liver steatosis, NAFLD, NASH and/or HCC are radiotherapy, chemotherapy, targeted drug therapy, immunotherapy and surgery.

In a further aspect, the sample is a biological sample from the subject. The sample may be any biological sample from the subject in which the aggregate and/or individual expression levels of PTPs and/or aggregate and/or individual oxidation levels of PTPs can be determined. In one embodiment, the biological sample is a tissue sample, a stool sample, a cell sample or a bodily fluid sample. In a further embodiment, the biological sample is a tissue sample, in particular a neoplastic tissue sample, such as a tumor sample. In a preferred embodiment, the sample is a liver tissue sample, in particular a liver tissue sample that is suspected of cancer. The biological sample may also be derived from a biological fluid or body fluid, for example, whole blood, blood, urine, lymph fluid, serum, plasma, nipple aspirate, ductal fluid, saliva, bile, sputum or tumor exudate.

In some embodiments of the methods of the present invention, the sample is a neoplastic tissue sample, or a tissue sample that is suspected to be neoplastic. In certain embodiment, the tissue sample is derived from fine-needle aspirate. In certain embodiments, the tissue sample is a resected tissue sample. In certain embodiments, the sample is a tissue biopsy or tissue fine-needle aspirate, for example a tumor tissue biopsy or tumor fine-needle aspirate from a primary tumor tissue or a metastatic tumor tissue. In other embodiments, the sample is resected tumor tissue, e.g. resected primary or metastatic tumor tissue. The biological sample can be obtained from the subject in any way typically used in clinical settings for obtaining a sample comprising the required cells or proteins. For example, the sample can be obtained from fresh, frozen, or paraffin-embedded surgical sample or biopsies of an organ or tissue comprising the suitable cells or proteins to be tested. If desired, the sample can be mixed with a fluid or purified or amplified or otherwise treated. For example, sample may be treated in one or more purification steps in order to increase the purity of the desired cells or proteins in the sample, or they may be examined without any purification steps.

In certain preferred embodiments, the sample may be a fresh sample or a frozen sample.

In particular embodiments, the presence and/or expression level of the aggregate PTPs, aggregate oxidized PTPs, and/or individual PTPs or oxidized PTPs are determined in a tissue sample, preferably a liver sample of the subject. Any existing, available or conventional separation, detection and quantification methods may be used herein to measure the presence or absence (e.g., readout being present vs. absent; or detectable amount vs. undetectable amount) and/or quantity (e.g., readout being an absolute or relative quantity, such as, for example, absolute or relative concentration) of markers, peptides, polypeptides, or proteins in samples.

For example, such methods may include mass spectrometry analysis methods, biochemical assay methods, immunoassay methods, or chromatography methods, or combinations thereof.

The term “immunoassay” generally refers to methods known as such for detecting one or more molecules or analytes of interest in a sample, wherein specificity of an immunoassay for the molecule(s) or analyte(s) of interest is conferred by specific binding between a specific-binding agent, commonly but without limitation an antibody, and the molecule(s) or analyte(s) of interest. Immunoassay technologies include without limitation immunohistochemistry, direct ELISA (enzyme-linked immunosorbent assay), indirect ELISA, sandwich ELISA, competitive ELISA, multiplex ELISA, radioimmunoassay (RIA), ELISPOT technologies, and other similar techniques known in the art.

Generally, any mass spectrometric (MS) techniques that are capable of obtaining precise information on the mass of peptides, and preferably also on fragmentation and/or (partial) amino acid sequence of selected peptides (e.g., in tandem mass spectrometry, MS/MS; or in post source decay, TOF MS), are useful herein. Suitable peptide MS and MS/MS techniques and systems are well-known per se (see, e.g., Methods in Molecular Biology, vol. 146: “Mass Spectrometry of Proteins and Peptides”, by Chapman, ed., Humana Press 2000, ISBN 089603609x; Biemann 1990. Methods Enzymol 193: 455-79; or Methods in Enzymology, vol. 402: “Biological Mass Spectrometry”, by Burlingame, ed., Academic Press 2005, ISBN 9780121828073) and may be used herein. MS peptide analysis methods may be advantageously combined with upstream peptide or protein separation or fractionation methods, such as for example with chromatography. The data obtained from MS may be processed using software for data analysis of proteomics and/or metabolomics known in the art, such as the Skyline software (v19.1.0.193).

Chromatography may also be used for measuring biomarkers. As used herein, the term “chromatography” encompasses methods for separating chemical substances, referred to as such and vastly available in the art. Chromatography as used herein may be preferably columnar (i.e., wherein the stationary phase is deposited or packed in a column), preferably liquid chromatography, and yet more preferably HPLC. While particulars of chromatography are well known in the art, for further guidance sec, e.g., Meyer M., 1998, ISBN: 047198373X, and “Practical HPLC Methodology and Applications”, Bidlingmeyer, B. A., John Wiley & Sons Inc., 1993.

Further peptide or polypeptide separation, identification or quantification methods may be used, optionally in conjunction with any of the above described analysis methods, for measuring biomarkers in the present disclosure. Such methods include, without limitation, chemical extraction partitioning, isoelectric focusing (IEF) including capillary isoelectric focusing (CIEF), capillary isotachophoresis (CITP), capillary electrochromatography (CEC), and the like, one-dimensional polyacrylamide gel electrophoresis (PAGE), two-dimensional polyacrylamide gel electrophoresis (2D-PAGE), capillary gel electrophoresis (CGE), capillary zone electrophoresis (CZE), micellar electrokinetic chromatography (MEKC), free flow electrophoresis (FFE), etc.

In preferred embodiments, said biomarkers can be determined using mass spectrometry analysis methods, biochemical assay methods, immunoassay methods, chromatography methods or combinations thereof. In a further preferred embodiment, they are determined using mass spectrometry analysis methods. In particular embodiments, the aggregate activity of PTPs and/or the presence and/or expression level of the individual PTPs and/or oxidized PTPs is determined using mass spectrometry, preferably liquid chromatography-mass spectrometry (LC-MS).

The person skilled in the art will understand that prior to mass spectrometry the biological sample may be depleted and processed, e.g. (ultra)filtrated, denaturated, alkylated, digested and/or deglycosylated.

As disclosed herein, the in vitro methods of the invention are provided to determine the aggregate expression level of PTPs and aggregate oxidation level of PTPs, and/or the presence and/or expression level of the individual PTPs and/or individual oxidized PTPs. In particular embodiments, said methods thus comprise measuring the quantity, amount or expression level of said biomarkers in the sample from the subject.

The terms “quantity”, “amount” and “level” are synonymous and generally well-understood in the art. The terms as used herein may particularly refer to an absolute quantification of a molecule or an analyte in a sample, or to a relative quantification of a molecule or analyte in a sample, i.e., relative to another value such as relative to a reference value as taught herein, or to a range of values indicating a base-line expression of the biomarker. These values or ranges can be obtained from a single patient or from a group of patients.

An absolute quantity of a molecule or analyte in a sample may be advantageously expressed as weight or as molar amount, or more commonly as a concentration, e.g., weight per volume or mol per volume. A relative quantity of a molecule or analyte in a sample may be advantageously expressed as an increase or decrease or as a fold-increase or fold-decrease relative to said another value, such as relative to a reference value as taught herein. Performing a relative comparison between first and second parameters (e.g., first and second quantities) may but need not require first to determine the absolute values of said first and second parameters. For example, a measurement method can produce quantifiable readouts (such as, e.g., signal intensities) for said first and second parameters, wherein said readouts are a function of the value of said parameters, and wherein said readouts can be directly compared to produce a relative value for the first parameter vs, the second parameter, without the actual need first to convert the readouts to absolute values of the respective parameters.

The terms “quantity”, and “expression level” of the biomarkers in the present application are used interchangeably in this specification to refer to the absolute and/or relative quantification, concentration level or amount of any such product in a sample. Preferably, the biomarker is a protein and the term “expression level” refers to the “protein expression level”.

Present inventors found that the aggregate expression level of PTPs in a biological sample is significantly different in patients suffering from HCC, compared to biological samples from healthy subjects or subjects with liver steatosis, NASH or NAFLD. Furthermore, the combination of the aggregate expression level of PTPs with the aggregate expression level of oxidized PTPs (also referred herein as the aggregate oxidation level of PTPs), and optionally with the individual expression levels of one or more PTPs or oxidised PTPs also provides biomarkers to identify HCC in subjects.

In particular embodiments, the method as taught herein may comprise the step of comparing the quantity or expression level of the aggregate PTPs, the aggregate oxidised PTPs and/or the individual expression level of one or more PTPs or oxidised PTPs in the sample of the subject with a reference or threshold quantity or expression level for said biomarkers. Said reference or threshold quantity or expression level may represent a known diagnosis of liver steatosis, NASH, NAFLD or HCC. It may also represent a healthy subject. Preferably, said reference threshold value is determined from healthy tissue.

In some embodiments, in vitro methods for assessing the efficacy of a therapeutic treatment of cancer, in particular HCC, in a subject are provided. In said methods, the presence of at least one of the biomarkers is evaluated in a tissue sample obtained at a time point before the start of the treatment and at regular intervals after the start of the treatment. Thereafter, the levels of said biomarkers in the first tissue samples are compared to the levels in one or more of the subsequent tissue samples, wherein a deviation or no deviation is indicative for the efficacy of the therapeutic treatment in the subject.

Distinct reference or threshold values may thus represent the diagnosis of cancer, more specifically HCC. In another example, distinct reference or threshold values may represent the need of a subject for a therapeutic treatment of cancer, in particular HCC. In another example, distinct reference or threshold values may be indicative for the selection of a prophylactic or therapeutic treatment of the subject. In still another example, distinct reference or threshold values may be indicative for the efficacy of the therapeutic treatment in the subject.

Such comparison may generally include any means to determine the presence or absence of at least one difference and optionally of the size of such difference between values or profiles being compared. A comparison may include a visual inspection, an arithmetical or statistical comparison of measurements. Such statistical comparisons include, but are not limited to, applying an algorithm. If the values or biomarker profiles comprise at least one standard, the comparison to determine a difference in said values or biomarker profiles may also include measurements of these standards, such that measurements of the biomarker are correlated to measurements of the internal standards.

Reference values or threshold values for the quantity or expression level of the aggregate PTPs, oxidized PTPs, and/or of the individual levels of PTPs or oxidised PTPs may be established according to known procedures previously employed for other biomarkers.

For example, a reference value of the aggregate expression level of PTPs or oxidised PTPs, and/or of the individual levels of PTPs or oxidised PTPs, for a particular diagnosis of HCC as taught herein may be established by determining the quantity or expression of said biomarker in a tissue sample(s) from one individual or from a population (e.g., group) of individuals characterized by said particular diagnosis of said disease or condition. Such population may comprise without limitation ≥2, ≥10, ≥100, or even several hundred individuals or more.

Hence, by means of an illustrative example, reference values of the aggregate expression level of PTPs or oxidised PTPs, and/or of the individual levels of PTPs or oxidised PTPs for the diagnosis of HCC vs. no such disease or condition may be established by determining the aggregate expression level of PTPs or oxidised PTPs, and/or of the individual levels of PTPs or oxidised PTPs, in a sample(s) from one individual or from a population of individuals diagnosed (e.g., based on other adequately conclusive means, such as, for example, clinical signs and symptoms, imaging, etc.) as, respectively having or not having HCC.

Measuring the quantity or expression level of aggregate expression level of PTPs or oxidised PTPs, and/or of the individual levels of PTPs or oxidised PTPs for the same patient at different time points may in such a case thus enable the continuous monitoring of the status of the patient and may lead to prediction of worsening or improvement of the patient's condition with regard to a given disease or condition as taught herein. Tools such as the kits described herein below can be developed to ensure this type of monitoring. One or more reference values, threshold values or ranges for said at least one biomarker quantities or expression levels linked to the development of HCC can, e.g., be determined beforehand or during the monitoring process over a certain period of time in said subject. Alternatively, these reference values or ranges can be established through data sets of several patients with highly similar disease phenotypes, e.g., from subjects not developing HCC. A sudden deviation of the levels of said at least one biomarker from said reference value, threshold value, or range can predict the worsening of the condition of the patient (e.g., at home or in the clinic) before the (often severe) symptoms actually can be felt or observed. Monitoring may be applied in the course of a medical treatment of a subject, preferably medical treatment aimed at alleviating the so-monitored disease or condition. Such monitoring may be comprised, e.g., in decision making whether a patient may be discharged, needs a change in treatment or needs further hospitalisation.

In an embodiment, reference value(s) or threshold value(s) as intended herein may convey absolute quantities of the biomarker as intended herein. In another embodiment, the quantity of the biomarker in a sample from a tested subject may be determined directly relative to the reference value (e.g., in terms of increase or decrease, or fold-increase or fold-decrease). Advantageously, this may allow the comparison of the quantity or expression level of the biomarker in the sample from the subject with the reference value (in other words to measure the relative quantity of the biomarker in the sample from the subject vis-à-vis the reference value) without the need first to determine the respective absolute quantities of the biomarker.

As explained, the present methods, uses, or products may involve finding a deviation or no deviation between the aggregate expression level of PTPs or oxidised PTPs, and/or of the individual levels of PTPs or oxidised PTPs as taught herein measured in a sample from a subject and a given reference value or threshold value.

A “deviation” of a first value from a second value or a “difference” between a first value and a second value may generally encompass any direction (e.g., increase: first value>second value; or decrease: first value<second value) and any extent of alteration.

For example, a deviation or a difference may encompass a decrease in a first value by, without limitation, at least about 10% (about 0.9-fold or less), or by at least about 20% (about 0.8-fold or less), or by at least about 30% (about 0.7-fold or less), or by at least about 40% (about 0.6-fold or less), or by at least about 50% (about 0.5-fold or less), or by at least about 60% (about 0.4-fold or less), or by at least about 70% (about 0.3-fold or less), or by at least about 80% (about 0.2-fold or less), or by at least about 90% (about 0.1-fold or less), relative to a second value with which a comparison is being made. For example, a deviation or a difference may encompass an increase of a first value by, without limitation, at least about 10% (about 1.1-fold or more), or by at least about 20% (about 1.2-fold or more), or by at least about 30% (about 1.3-fold or more), or by at least about 40% (about 1.4-fold or more), or by at least about 50% (about 1.5-fold or more), or by at least about 60% (about 1.6-fold or more), or by at least about 70% (about 1.7-fold or more), or by at least about 80% (about 1.8-fold or more), or by at least about 90% (about 1.9-fold or more), or by at least about 100% (about 2-fold or more), or by at least about 150% (about 2.5-fold or more), or by at least about 200% (about 3-fold or more), or by at least about 500% (about 6-fold or more), or by at least about 700% (about 8-fold or more), or like, relative to a second value with which a comparison is being made.

Preferably, a deviation or a difference may refer to a statistically significant observed alteration. For example, a deviation or a difference may refer to an observed alteration which falls outside of error margins of reference values in a given population (as expressed, for example, by standard deviation or standard error, or by a predetermined multiple thereof, e.g., ±1×SD or ±2×SD or ±3×SD, or ±1×SE or ±2×SE or ±3×SE). Deviation or a difference may also refer to a value falling outside of a reference range defined by values in a given population (for example, outside of a range which comprises ≥40%, ≥50%, ≥60%, 270%, 275% or 280% or ≥85% or ≥90% or ≥95% or even ≥100% of values in said population).

In a further embodiment, a deviation or a difference may be concluded if an observed alteration is beyond a given threshold or cut-off. Such threshold or cut-off may be selected as generally known in the art to provide for a chosen accuracy, sensitivity and/or specificity of the prediction methods, e.g., accuracy, sensitivity and/or specificity of at least 50%, or at least 60%, or at least 70%, or at least 80%, or at least 85%, or at least 90%, or at least 95%.

For example, receiver-operating characteristic (ROC) curve analysis can be used to select an optimal threshold or cut-off value of the quantity of a given biomarker for clinical use of the present diagnostic tests, based on acceptable global accuracy, sensitivity and/or specificity, or related performance measures which are well-known per se, such as positive predictive value (PPV), negative predictive value (NPV), positive likelihood ratio (LR+), negative likelihood ratio (LR−), Youden index, or similar. For example, an optimal threshold or cut-off value may be selected for each individual biomarker as a local extremum of the receiver operating characteristic (ROC) curve, i.e. a point of local maximum distance to the diagonal line, as described in Robin X., PanelomiX: a threshold-based algorithm to create panels of biomarkers, 2013, Translational Proteomics, 1(1):57-64.

The person skilled in the art will understand that it is not relevant to give an exact threshold or cut-off value. A relevant threshold or cut-off value can be obtained by correlating the sensitivity and specificity and the sensitivity/specificity for any threshold or cut-off value.

It is to the diagnostic engineers to determine which level of positive predictive value/negative predictive value/sensitivity/specificity is desirable and how much loss in positive or negative predictive value is tolerable. The chosen threshold or cut-off level could be dependent on other diagnostic parameters used in combination with the present method by the diagnostic engineers.

The present methods, uses, or products may further involve attributing any finding of a deviation or no deviation between the quantity or expression level of the PTPs or oxidised PTPs, and/or of the individual levels of PTPs or oxidised PTPs as taught herein measured in a tissue sample from a subject and a given reference value or threshold to the presence or absence of HCC.

In the methods provided herein the observation of a deviation between the aggregate expression level of PTPs or oxidised PTPs, and/or of the individual levels of PTPs or oxidised PTPs in a biological sample from a subject and a reference value or threshold value can lead to the conclusion that the diagnosis of cancer, in particular HCC, in said subject is different from that represented by said reference value or threshold value. Similarly, when no deviation is found between aggregate expression level of PTPs or oxidised PTPs, and/or of the individual levels of PTPs or oxidised PTPs in a biological sample from the subject and a reference value or threshold value, the absence of such deviation can lead to the conclusion that the diagnosis of cancer in said subject is substantially the same as that represented by said reference value or threshold value.

In particular embodiments, the reference value or threshold value as used in the methods according to the invention is determined from a biological sample from a subject or a group of subjects not affected by cancer, such as a healthy subject or a group of healthy subjects. The healthy subject or group of healthy subjects may be at high or average risk of developing cancer such as HCC, for example, the healthy subject or group of healthy subjects may be a subject with an early stage of liver steatosis. The quantity or expression level of at least one of the biomarkers in a biological sample from a subject, e.g. a subject with HCC, may be elevated (e.g., if the biomarker is oxidised PTPN1 or oxidised PTPN2) or decreased (e.g., if the biomarker is the aggregate expression level of PTPs) compared to (i.e., relative to) a reference value or threshold value representing the aggregate expression level of PTPs or oxidised PTPs, and/or of the individual levels of PTPs or oxidised PTPs in a biological sample from a subject or a group of subjects not affected by HCC, such as a healthy subject or a group of healthy subjects, or from a subject suffering from liver steatosis, NASH or NALFD. The so-elevated or decreased quantity or expression level may allow for the diagnosis of HCC in the subject.

The term “subject” or “patient” as used herein typically and preferably denotes humans, but may also encompass reference to non-human animals, preferably warm-blood animals, more preferably vertebrates, even more preferably mammals, such as, e.g. non-human primates, rodents, canines, felines, equines, oyines, porcines, and the like. Particularly intended are subjects known or suspected to have cancer, in particular HCC. Suitable subjects may include ones presenting to a physician for a screening for liver steatosis, NAFLD, NASH or HCC, and/or with symptoms and signs indicative of liver steatosis, NAFLD, NASH or HCC.

In particular embodiments, the subject is a subject at risk of cancer, such as a subject at average or high risk of HCC. Non-limiting examples of risk factor for HCC include obesity, alcohol use, genetic susceptibility, diet.

In more particular embodiments, the subject is a subject with obesity.

The diagnostic method as taught herein can be used to efficiently complement imaging techniques in cancer screening, such as HCC screening.

In particular embodiments, the subject is a subject diagnosed with liver steatosis, NAFLD, NASH or HCC, for example by imaging techniques.

The present methods of the diagnosis, prognosis or monitoring of cancer such as HCC may be adequately qualified as in vitro methods in that they apply one or more in vitro processing and/or analysis steps to a sample removed from the subject; preferably to a liver tissue sample removed from the subject. The term “in vitro” generally denotes outside, or external to, a body, e.g., an animals or human body. Detecting or measuring the aggregate expression level of PTPs or oxidised PTPs, and/or of the individual levels of PTPs or oxidised PTPs in a tissue sample from a subject may ordinarily imply that the examination phase of the present methods comprise measuring the quantity or presence of at least one of said biomarkers in the sample from the subject. One understands that the present methods may generally comprise an examination phase in which data is collected from and/or about the subject.

In a further aspect, the application provides a kit for diagnosing, predicting, prognosing and/or monitoring cancer, such as HCC, in a subject, or for assisting in the selection of a prophylactic or therapeutic treatment of cancer in a subject, or for assessing the efficacy of a therapeutic treatment of cancer in a subject, said kit comprising: means for measuring the aggregate expression level of PTPs in a sample of the subject, means for measuring the aggregate oxidation level of PTPs in the sample of the subject, and a reference value or threshold value of the aggregate expression level of PTPs, and of the aggregate oxidation level of PTPs, or means for establishing said reference value or threshold value, wherein said reference value or threshold represents a known diagnosis, prediction and/or prognosis of cancer, in particular HCC, such as wherein said reference value or threshold value corresponds to the aggregate expression of PTPs and to the aggregate oxidation level of PTPs, in a sample not affected by cancer, such as in a healthy tissue, or wherein said reference value or threshold value corresponds to the aggregate expression level of PTPs, and the aggregate oxidation level of PTPs, in a tissue affected by cancer.

In some embodiments, the kit may comprise means for measuring the aggregate expression level of all PTPs in a sample of the subject, means for measuring the aggregate oxidation level of all PTPs in the sample of the subject, and a reference value or threshold value of said aggregate expression level and said aggregate oxidation level, or means for establishing said reference value or threshold value, wherein said reference value or threshold value represents a known diagnosis, prediction and/or prognosis of cancer, such as wherein said reference value or threshold value corresponds to the aggregate expression level and the aggregate oxidation level of all PTPs in a sample not affected by cancer, such as in a healthy sample, or wherein said reference value or threshold value corresponds to the aggregate expression level and the aggregate oxidation level of all PTPs in a sample affected by cancer.

In some embodiments, the kit as taught herein comprises means for measuring the aggregate expression level of a group of PTPs at least comprising PTPN2, PTPRF, PTPRK, PTPN12, PTPN7, PTPN1, and PTPN6 in a sample of the subject, means for measuring the aggregate oxidation level of a group of PTPs at least comprising PTPN2, PTPRF, PTPRK, PTPN12, PTPN7, PTPN1, and PTPN6 in a sample of the subject; and a reference value or threshold value of said aggregate expression level and said aggregate oxidation level, or means for establishing said reference value or threshold value, wherein said reference value or threshold value represents a known diagnosis, prediction and/or prognosis of cancer, such as wherein said reference value or threshold value corresponds to the aggregate expression level and the aggregate oxidation level of a group of PTPs at least comprising PTPN2, PTPRF, PTPRK, PTPN12, PTPN7, PTPN1, and PTPN6 in a sample not affected by cancer, such as in a healthy sample, or wherein said reference value or threshold value corresponds to the aggregate expression level and the aggregate oxidation level of a group of PTPs at least comprising PTPN2, PTPRF, PTPRK, PTPN12, PTPN7, PTPN1, and PTPN6 in a sample affected by cancer.

In some embodiments, the kit as taught herein comprises means for measuring the aggregate expression level of a group of PTPs at least comprising PTPRB, PTPRD, PTPRF, PTPRM, PTPRG, PTPRK, PTPRJ, PTPRC, PTPRA, PTPRE, PTPN1, PTPN2, PTPN3, PTPN6, PTPN7, PTPN9, PTPN11 and PTPN12 in a sample of the subject, means for measuring the aggregate oxidation level of a group of PTPs at least comprising PTPRB, PTPRD, PTPRF, PTPRM, PTPRG, PTPRK, PTPRJ, PTPRC, PTPRA, PTPRE, PTPN1, PTPN2, PTPN3, PTPN6, PTPN7, PTPN9, PTPN11 and PTPN12 in the sample of the subject; and a reference value or threshold value of said aggregate expression level and said aggregate oxidation level, or means for establishing said reference value or threshold value, wherein said reference value or threshold value represents a known diagnosis, prediction and/or prognosis of cancer, such as wherein said reference value or threshold value corresponds to the aggregate expression level and the aggregate oxidation level of a group of PTPs at least comprising PTPRB, PTPRD, PTPRF, PTPRM, PTPRG, PTPRK, PTPRJ, PTPRC, PTPRA, PTPRE, PTPN1, PTPN2, PTPN3, PTPN6, PTPN7, PTPN9, PTPN11 and PTPN12 in a sample not affected by cancer, such as in a healthy sample, or wherein said reference value or threshold value corresponds to the aggregate expression level and the aggregate oxidation level of a group of PTPs at least comprising PTPRB, PTPRD, PTPRF, PTPRM, PTPRG, PTPRK, PTPRJ, PTPRC, PTPRA, PTPRE, PTPN1, PTPN2, PTPN3, PTPN6, PTPN7, PTPN9, PTPN11 and PTPN12 in a sample affected by cancer.

In some embodiments, the kit further comprises means for measuring the expression level of one or more individual PTPs in the sample of the subject, and a reference or threshold value of the expression value of the one or more individual PTPs or means for establishing said reference value or threshold value, wherein said reference value or threshold value represents a known diagnosis, prediction and/or prognosis of cancer, such as wherein said reference value or threshold value corresponds to the expression level of one or more individual PTPs in a sample not affected by cancer, such as in a healthy sample, or wherein said reference value or threshold value corresponds to the expression level of the one or more individual PTPs in a sample affected by cancer.

In some embodiments, the kit further comprises means for measuring the expression level of one or more individual oxidized PTPs in the sample of the subject, and a reference value or threshold value of the expression level of the one or more individual oxidized PTPs or means for establishing said reference value or threshold value, wherein said reference value or threshold value represents a known diagnosis, prediction and/or prognosis of cancer, such as wherein said reference value or threshold value corresponds to the expression level of the one or more individual oxidized PTPs, in a sample not affected by cancer, such as in a healthy sample, or wherein said reference value or threshold value corresponds to the expression level of the one or more individual oxidized PTPs in a sample affected by cancer.

Various techniques for measuring biomarkers may employ binding agents for said respective biomarkers. Hence, the means for measuring the aggregate expression level of PTPs or oxidised PTPs, and/or the expression level of one or more individual PTPs and/or oxidised PTPs in a sample from a subject may comprise binding agents capable of specifically binding to said biomarkers, e.g. an antibody or antibody fragment, aptamer, photoaptamer, protein, peptide, peptidomimetic, or a small molecule, and/or carriers which allow visualization and/or a qualitative read-out of the measurement, for example, by spectrophotometry. In some embodiments, binding agents as taught herein may comprise a detectable label. In some embodiments, binding agents may be provided with a tag that permits detection with another agent (e.g., with a probe binding partner). The biomarker-binding agent conjugate may be associated with or attached to a detection agent to facilitate detection.

In particular embodiments, the reference value or threshold value is a reference value or threshold value for the aggregate expression level of PTPs or oxidised PTPs, and/or the expression level of one or more individual PTPs and/or oxidised PTPs, wherein said reference value or threshold value corresponds to the quantity or expression level of said biomarkers in a sample from a subject not affected by cancer, such as a sample from a healthy subject or a group of healthy subjects, or wherein said reference value or threshold value corresponds to the quantity or expression level of said biomarkers in a sample from a subject or a group of subjects affected by cancer, such as HCC.

In particular embodiments, the kit further comprises a reference or threshold score representing the number of biomarkers selected from the aggregate expression level of PTPs or oxidised PTPs, and/or the expression level of one or more individual PTPs and/or oxidised PTPs that were found to deviate from their respective reference value or threshold value.

The kit for diagnosing cancer in a subject may further comprise ready-to use substrate solutions, wash solutions, dilution buffers and instructions. The diagnostic kit may also comprise positive and/or negative control samples.

Preferably, the instructions included in the diagnostic kit are unambiguous, concise and comprehensible to those skilled in the art. The instructions typically provide information on kit contents, how to collect the tissue sample, methodology, experimental read-outs and interpretation thereof and cautions and warnings.

The terms “kit of parts” and “kit” as used throughout this specification refer to a product containing components necessary for carrying out the specified methods (e.g., methods for the diagnosis cancer in a subject or for determining whether a subject is in need of therapeutic treatment of cancer as taught herein), packed so as to allow their transport and storage. Materials suitable for packing the components comprised in a kit include crystal, plastic (e.g., polyethylene, polypropylene, polycarbonate), bottles, flasks, vials, ampules, paper, envelopes, or other types of containers, carriers or supports. Where a kit comprises a plurality of components, at least a subset of the components (e.g., two or more of the plurality of components) or all of the components may be physically separated, e.g., comprised in or on separate containers, carriers or supports. The components comprised in a kit may be sufficient or may not be sufficient for carrying out the specified methods, such that external reagents or substances may not be necessary or may be necessary for performing the methods, respectively. Typically, kits are employed in conjunction with standard laboratory equipment, such as liquid handling equipment, environment (e.g., temperature) controlling equipment, analytical instruments, etc. In addition to the recited binding agents(s) as taught herein, such as for example, antibodies, hybridisation probes, amplification and/or sequencing primers, optionally provided on arrays or microarrays, the present kits may also include some or all of solvents, buffers (such as for example but without limitation histidine-buffers, citrate-buffers, succinate-buffers, acetate-buffers, phosphate-buffers, formate buffers, benzoate buffers, TRIS (Tris(hydroxymethyl)-aminomethan) buffers or maleate buffers, or mixtures thereof), enzymes (such as for example but without limitation thermostable DNA polymerase), detectable labels, detection reagents, and control formulations (positive and/or negative), useful in the specified methods. Typically, the kits may also include instructions for use thereof, such as on a printed insert or on a computer readable medium. The terms may be used interchangeably with the term “article of manufacture”, which broadly encompasses any man-made tangible structural product, when used in the present context.

In particular embodiments, the kit further comprises a computer readable storage medium having recorded thereon one or more programs for carrying out the method as taught herein.

Further, also the use of such a kit is provided, in particular for diagnosis, prediction, prognosis and/or monitoring of cancer in a subject, or for assisting in the selection of a prophylactic or therapeutic treatment of cancer in a subject, or for assessing the efficacy of a therapeutic treatment of cancer in a subject.

In still another aspect, a therapeutic or prophylactic agent for use as a medicament in the treatment of treatment of cancer is provided, wherein said agent is capable of modulating, such as inhibiting or increasing, expression or activity of at least the aggregate expression level of PTPs and/or oxidised PTPs, and/or one or more of the individual PTPs and/or oxidised PTPs

Further, the methods, the kit or the agent for use according to any of the embodiments of the invention are directed to cancer, in particular HCC. In a further embodiment, said HCC is obesity-induced HCC.

In a further aspect, provided herein is a method of diagnosing and treating cancer, in particular HCC, in a subject comprising:
(a) Determining the presence and/or expression level of the aggregate PTPs and the aggregate oxidised PTPs,
(b) Diagnosing cancer in the subject based on the aggregate expression level of PTPs and the aggregate oxidation level of PTPs, and
(c) Administering to said subject diagnosed with cancer a therapy for cancer, such as administering to said subject an effective amount of a therapeutic agent for cancer.

In some further embodiments, the method of diagnosing and treating cancer, in particular HCC, in a subject, further comprises determining the presence and/or the expression level of one or more individual PTPs and/or oxidised PTPs in a biological sample from the subject, diagnosing cancer in the subject based on the presence and/or the expression level of one or more individual PTPs and/or oxidised PTPs, and administering to said subject diagnosed with cancer a therapy for cancer, such as administering to said subject an effective amount of a therapeutic agent for cancer.

It is apparent that there have been provided in accordance with the invention products, methods, and uses, that provide for substantial advantages as set forth above. While the invention has been described in conjunction with specific embodiments thereof, it is evident that many alternatives, modifications, and variations will be apparent to those skilled in the art in light of the foregoing description. Accordingly, it is intended to embrace all such alternatives, modifications, and variations as follows in the spirit and broad scope of the appended claims.

The above aspects and embodiments are further supported by the following non-limiting examples.

### EXAMPLES

### Materials and Methods

### Tissue Homogenisation

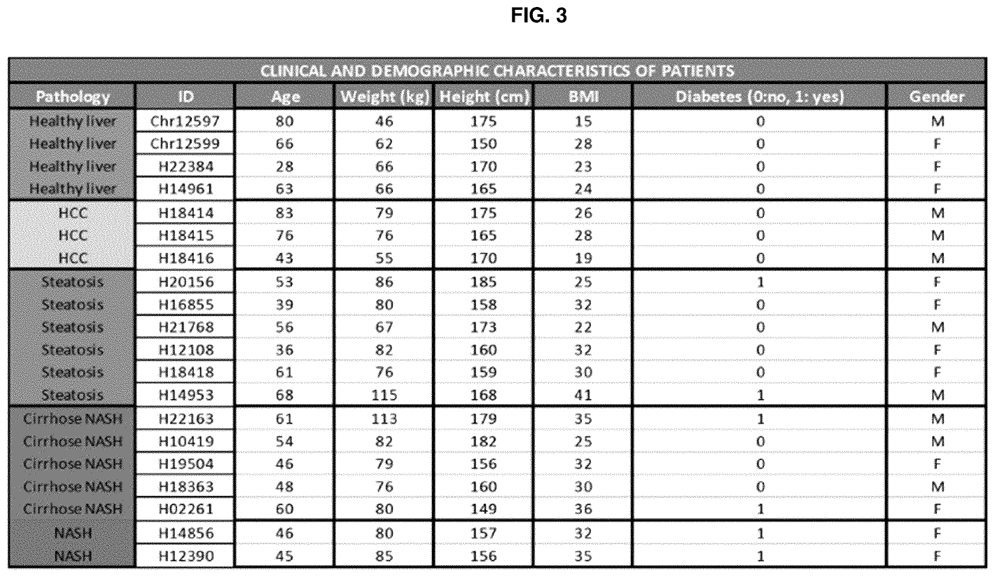

Fresh frozen liver tissue specimen were collected from several patients diagnosed with the different stages of liver diseases and of healthy subjects (FIG. 3) and tissue samples were homogenized according to the following protocol:
Transfer fresh frozen tissue specimen to Eppendorf tube.
Add equal weight of acid-washed glass beads in degassed lysis buffer (10% glycerol, 1% NP-40, 1× protease inhibitor, 1× phosphatase inhibitor, in PBS).
Disrupt tissue by beads-beating at 4 degrees for 10 min, with 30 s on/off cycles.
Sonicate for 10 min at 4 degrees, with 30 s on/off cycles.
Pellet debris at 20,000×g for 1 h at 4 degrees.
Retain supernatant for estimation of protein content by Bradford against BSA standard curve.

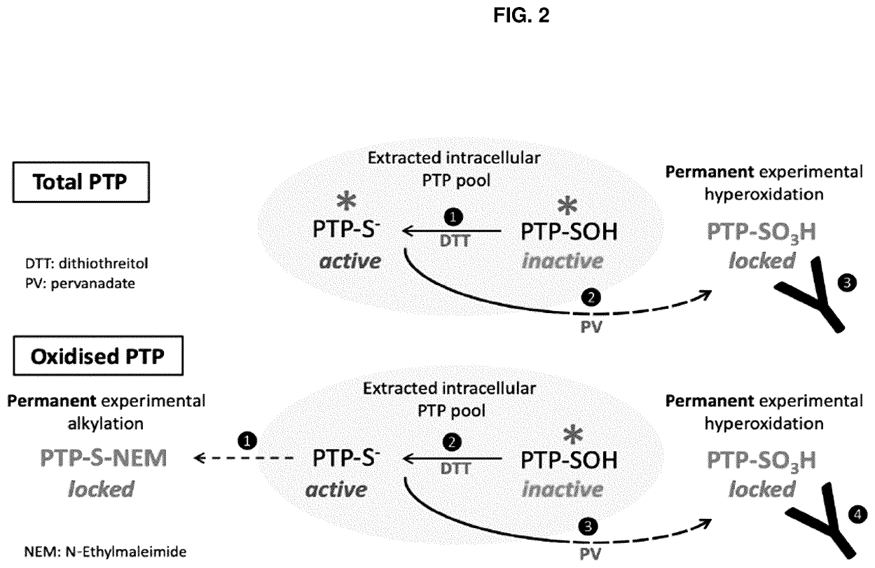

### Chemical Derivatisation (FIG. 2)

Reduction of oxidised PTPs with 10 mM Dithiothreitol in lysis buffer, 1 h at 20 degrees.
Alkylation of reduced PTPs with 20 mM N-Ethylmaleimide in lysis buffer, 30 min at 20 degrees in the dark.
Hyperoxidation of reduced PTPs with 1 mM pervanadate in lysis buffer for 30 min in the dark.

### Lysate Digestion

Reduce total proteins in 4 mM Dithiothreitol for 1 h at 20 degrees.
Alkylate total proteins in 8 mM Iodoacetamide for 30 min at 20 degrees, in the dark.
Quench Iodoacetamide with another 4 mM Dithiothreitol, mix and proceed.
Per 50 μg of total protein, add 1 ug of Lys C, incubate for 4 h at 37 degrees.
Per 50 ug of total protein, add 1 ug of Trypsin, incubate for 16 h at 37 degrees.
Quench digestion with formic acid to 0.1% final concentration, snap freeze for subsequent use.

### PTP Catalytic Peptide Pulldown

Dilute digested lysate in PBS, pellet at 20,000×g for 10 min at 4 degrees. Retain soluble fraction for pulldown as input.
Per sample, perform pulldown overnight with end-to-end incubation at 4 degrees, with 10 ul of Protein A/G agarose beads coated with 10 ug of oxPTP antibody (R&D Systems, MAB2844).
After overnight incubation, retrieve agarose beads and retain IP flowthrough for tissue proteome profiling (requires also SCX micro STAGE tip clean up, as below).
Wash agarose beads 3 times with PBS, and 3 times with MS grade water.
Elute with 10% TFA, 90% MS grade water, dry peptides by vacuum centrifugation.
Perform clean-up on eluted peptides by home-made strong cation exchange (SCX) micro STAGE tips, (steps a-d below).
a. Clean SCX STAGE tip with 100% Methanol. Equilibrate with 80% MS grade water, 20% ACN, 0.1% formic acid.
b. Reconstitute dry peptides in 80% MS grade water, 20% ACN, 0.1% formic acid for SCX loading.
c. Wash SCX STAGE tip with 80% MS grade water, 20% ACN, 0.1% formic acid.
d. Elute SCX STAGE tip with 80% MS grade water, 20% ACN, 0.1% formic acid, 0.5M Ammonium acetate.
Dry eluate by vacuum centrifugation, ready for MS injection.

### LC-MS/MS Analysis of PTP Catalytic Peptides

Parameters for PTP peptide separation, identification and quantitation by Agilent 1290 LC system, coupled to Thermo Scientific Q Exactive HF-X mass spectrometer.
LC Buffer A=100% MS grade water, 0.1% formic acid; LC Buffer B=80% ACN, 20% MS grade water, 0.1% formic acid. Linear elution gradient from 10 to 44% Buffer B in 25 min gradient time (total 40 min analysis time).
Perform mass spectrometry acquisitions in data dependent acquisition mode. With the following settings:

FULL MS/DD-MS² (TOPN)
General

| Runtime | 0 to 40 | min |
|---|---|---|
| Polarity | positive | |
| In-source CID | 0.0 | eV |

| Default charge state | 2 |
|---|---|
| Inclusion | — |
| Exclusion | — |
| Tags | — |

Full MS

| Microscans | 1 | |
|---|---|---|
| Resolution | 60,000 | |
| AGC target | 3e6 | |
| Maximum IT | 20 | ms |
| Number of scan ranges | 1 | |
| Scan range | 375 to 1600 | m/z |

| | |
|---|---|
| Spectrum data type | Profile |

dd-MS²/dd-SIM

| Microscans | 1 | |
|---|---|---|
| Resolution | 15,000 | |
| AGC target | 1e5 | |
| Maximum IT | 150 | ms |
| Loop count | 7 | |
| MSX count | 1 | |
| TopN | 7 | |
| Isolation window | 1.4 | m/z |
| Isolation offset | 0.0 | m/z |
| Scan range | 200 to 2000 | m/z |
| Fixed first mass | 120.0 | m/z |

| | |
|---|---|
| (N) CE/stepped (N) CE | nce: 27 |
| Spectrum data type | Profile |

dd Settings

| Minimum AGE target | 1.00e4 | |
|---|---|---|
| Intensity threshold | 6.7e4 | |
| Apex trigger | — | |
| Charge exclusion | unassigned, 1, 6-8, >8 | |
| Multiple charge states | all | |
| Peptide match | preferred | |
| Exclude isotopes | on | |
| Dynamic exclusion | 6.0 | s |

| | |
|---|---|
| If idle . . . | do not pick others |

Database search against current human Uniprot database.
Manual spectral inspection needed to confirm good ion ladder coverage and confident localisation of hyperoxidised cysteine.
Quantification by area under curve, or semi-quantification by spectral counting.

### Results

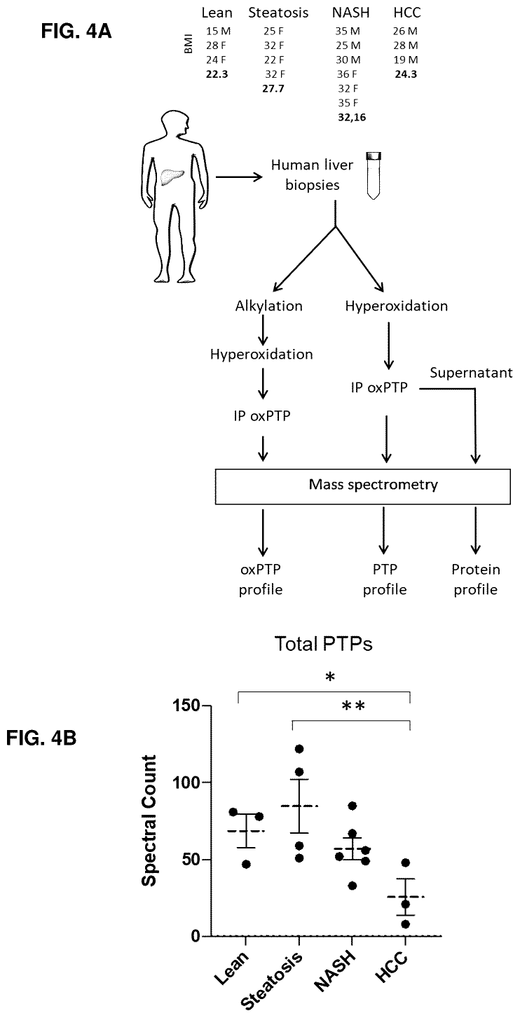

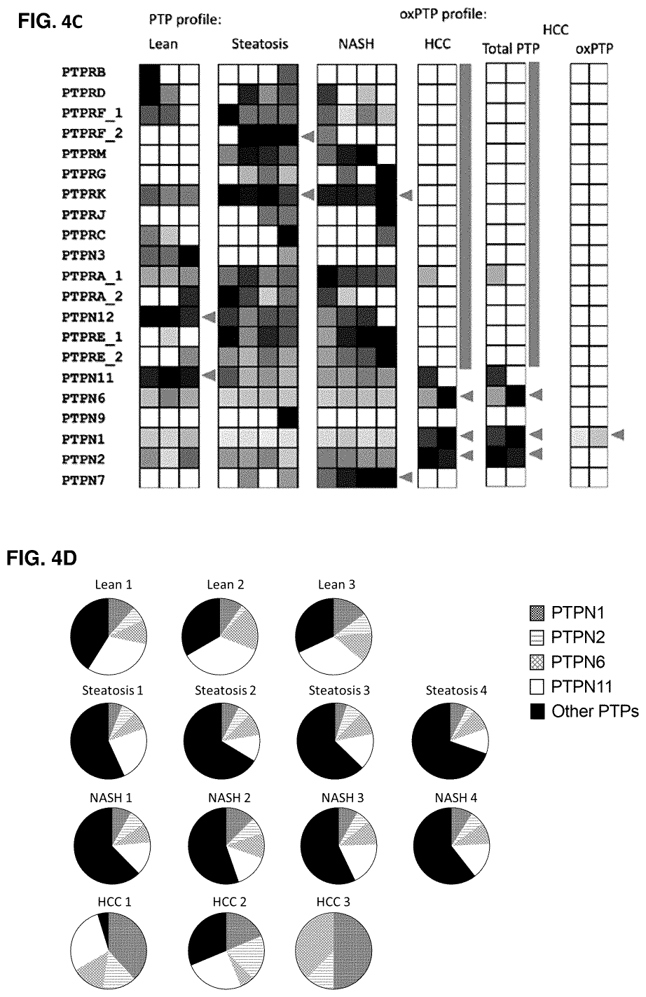

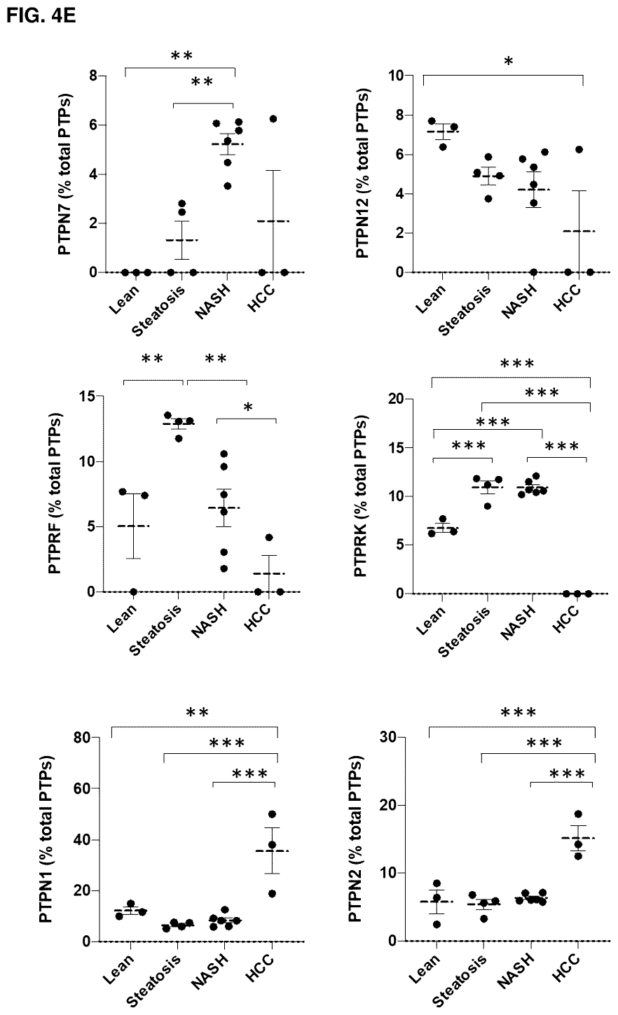

To characterise a global PTP profile representing the aggregated activity of PTPs of liver biopsy samples, we took advantage of a quantitative proteomics approach described in detail in the materials and methods section herein above and summarized in FIG. 4A, to assess for the first time aggregate PTP expression/oxidation in human biopsies (FIG. 4A). Briefly, global or aggregate PTP expression and oxidation is assessed in snap-frozen biopsies, samples have been divided in two sets and homogenized in the presence of reagents to alkylate/hyperoxidate all PTPs that are not irreversibly oxidized in one hand, and only reagents to hyperoxidate all PTPs in the other hand. PTPs are immunoprecipitated taken advantage of a specific antibody developed against the oxidised catalytic site of all PTPs. Importantly, with the supernatant we run a proteomic analysis to assess all proteins present in the sample. This approach allowed us to define 1) the complete list of present PTPs, 2) the degree of oxidation of individual PTPs, and 3) the complete or aggregate set of PTP proteins present in the samples. The first screening and set up of the technique was performed with the selected samples described in FIG. 4A. Proteome data from the biopsies show reduced aggregate PTP expression in HCC samples but not global differences in the TyrPhome of the lean/steatosis/NASH samples (FIG. 4B). We observed differences in individual PTP expression between the groups (FIGS. 4C-E) and oxidation of PTPN1 in the HCC samples (FIG. 4C). Our results thus show the identification of HCC samples using the aggregate PTPs levels, and the identification of individual PTPs that can contribute to the transition from lean, NAFLD, NASH to HCC (e.g. PTPRF, PTPRK, PTPN7, PTPN12, PTPN2, PTPN1) and oxidation of PTPN1 in HCC samples.

### REFERENCES

Grohmann, M. et al. (2018) Obesity Drives STAT-1-Dependent NASH and STAT-3-Dependent HCC. Cell 175 (5), 1289-1306 e20.
Gurzov, E. N. et al. (2014) Hepatic oxidative stress promotes insulin-STAT-5 signaling and obesity by inactivating protein tyrosine phosphatase N2. Cell Metab 20 (1), 85-102.
Gurzov, E. N. et al. (2015) Protein tyrosine phosphatases: molecular switches in metabolism and diabetes. Trends Endocrinol Metab 26 (1), 30-9.
Karisch R. et al. (2011) Global proteomic assessment of the classical protein-tyrosine phosphatome and “Redoxome. Cell 146(5): 826-40.
Litwak, S. A. et al. (2017) JNK Activation of BIM Promotes Hepatic Oxidative Stress, Steatosis, and Insulin Resistance in Obesity. Diabetes 66 (12), 2973-2986.
Tonks, N. K. (2013) Protein tyrosine phosphatases—from housekeeping enzymes to master regulators of signal transduction. FEBS J 280 (2), 346-78.
Wu, W. et al. (2017) Differential oxidation of protein-tyrosine phosphatases during zebrafish caudal fin regeneration. Sci Rep. 7(1), 12699.

1. An in vitro method for measuring protein tyrosine phosphatases (PTPs) in a subject, the method comprising:
measuring the aggregate expression level of PTPs in a sample of the subject, and
measuring the aggregate oxidation level of PTPs in a sample of the subject,
wherein the aggregate expression level and the aggregate oxidation level correspond to a reference value or threshold value that is characteristic of a subject with a known diagnosis, prognosis and/or disease status of cancer, and wherein a deviation or no deviation is indicative for the diagnosis, prognosis and/or disease status of cancer in the subject.

2. (canceled)

3. An in vitro method for the prophylactic or therapeutic treatment of cancer in a subject, the method comprising:
treating the subject with a selected prophylactic or therapeutic treatment for cancer,
wherein the prophylactic or therapeutic treatment for cancer has been selected by:
measuring the aggregate expression level of protein tyrosine phosphatases (PTPs) in a sample of the subject,
measuring the aggregate oxidation level PTPs in a sample of the subject, and
comparing the aggregate expression level and the aggregate oxidation level to a corresponding reference value or threshold value that is characteristic of a subject with a known diagnosis, prognosis and/or disease status of cancer, wherein a deviation or no deviation is indicative for the selection of the prophylactic or therapeutic treatment of the subject.

4. An in vitro method for assessing a therapeutic treatment of cancer in a subject, the method comprising:
measuring the aggregate expression level of protein tyrosine phosphatases (PTPs) in a sample of the subject at a time point before the start of the treatment and one or more time points after the start of the treatment,
measuring the aggregate oxidation level PTPs in a sample of the subject at a time point before the start of the treatment and one or more time points after the start of the treatment, and
wherein the aggregate expression level and the aggregate oxidation level in the first sample with the aggregate expression level and said aggregate oxidation level in one or more of the subsequent samples, wherein a deviation or no deviation is indicative for the efficacy of the therapeutic treatment in the subject.

5. The method according to claim 1, wherein the aggregate expression level of all PTPs and the aggregate oxidation level of all PTPs present in the sample is determined.

6. The method according to claim 1, wherein the aggregate expression level and the aggregate oxidation level of PTPs is determined in a group of PTPs comprising at least PTPN2, PTPRF, PTPRK, PTPN12, PTPN7, PTPN1, and PTPN6.

7. The method according to claim 1, wherein the aggregate expression level and the aggregate oxidation level of PTPs is determined in a group of PTPs comprising at least PTPRB, PTPRD, PTPRF, PTPRM, PTPRG, PTPRK, PTPRJ, PTPRC, PTPRA, PTPRE, PTPN1, PTPN2, PTPN3, PTPN6, PTPN7, PTPN9, PTPN11 and PTPN12.

8. The method according to claim 1, further comprising determining the expression level of one or more individual PTPs and/or the expression level of one or more individual oxidised PTPs, and wherein the expression level of one or more individual PTPs and/or one or more individual oxidised PTPs corresponds to a reference value or threshold value that is characteristic of a subject with a known diagnosis, prognosis and/or disease status of cancer, wherein a deviation or no deviation is indicative for the diagnosis, prognosis and/or disease status of the subject, or indicative for the risk of developing cancer, or indicative for the selection of the prophylactic or therapeutic treatment of the subject, or indicative for the efficacy of the therapeutic treatment in the subject.

9. The in vitro method according to claim 8 wherein the individual PTPs and/or individual oxidized PTPs are selected from PTPRB, PTPRD, PTPRF, PTPRM, PTPRG, PTPRK, PTPRJ, PTPRC, PTPRA, PTPRE, PTPN1, PTPN2, PTPN3, PTPN6, PTPN7, PTPN9, PTPN11 and PTPN12.

10. The in vitro method according to claim 8, wherein the expression level of at least 2 of the individual PTPs and/or individual oxidised PTPs is determined.

11. The in vitro method according to claim 8, wherein the expression level of PTPN2, PTPRF, PTPRK, PTPN12, PTPN7, PTPN1, and PTPN6 is determined.

12. The in vitro method according to claim 8, wherein the expression level of oxidized PTPN1, oxidized PTPN2 and oxidized PTPN6 is determined.

13. (canceled)

14. (canceled)

15. (canceled)

16. (canceled)

17. A kit for diagnosing, predicting, prognosing and/or monitoring cancer in a subject, for determining the risk of developing cancer in a subject, or for assisting in the selection of a prophylactic or therapeutic treatment of cancer in a subject, or for assessing the efficacy of a therapeutic treatment of cancer in a subject, the kit comprising:
means for measuring the aggregate expression level of PTPs in a sample of the subject;
means for measuring the aggregate oxidation level of PTPs in the sample of the subject; and
a reference value or threshold value of the aggregate expression level and the aggregate oxidation level, or means for establishing the reference value or threshold value, wherein the reference value or threshold value represents a known diagnosis, prediction and/or prognosis of cancer, or wherein the reference value or threshold value corresponds to the aggregate expression level and the aggregate oxidation level of PTPs in a sample not affected by cancer, or wherein the reference value or threshold value corresponds to the aggregate expression level and the aggregate oxidation level of PTPs in a sample affected by cancer.

18. The kit of claim 17 further comprising:
means for measuring the aggregate expression level of all PTPs in a sample of the subject;
means for measuring the aggregate oxidation level of all PTPs in the sample of the subject; and
a reference value or threshold value of the aggregate expression level and said aggregate oxidation level, or means for establishing the reference value or threshold value, wherein the reference value or threshold value represents a known diagnosis, prediction and/or prognosis of cancer, or wherein the reference value or threshold value corresponds to the aggregate expression level and the aggregate oxidation level of all PTPs in a sample not affected by cancer, or wherein the reference value or threshold value corresponds to the aggregate expression level and the aggregate oxidation level of all PTPs in a sample affected by cancer.

19. The kit of claim 17 further comprising:
means for measuring the aggregate expression level of a group of PTPs at least comprising PTPN2, PTPRF, PTPRK, PTPN12, PTPN7, PTPN1, and PTPN6 in a sample of the subject;
means for measuring the aggregate oxidation level of a group of PTPs at least comprising PTPN2, PTPRF, PTPRK, PTPN12, PTPN7, PTPN1, and PTPN6 in the sample of the subject; and
a reference value or threshold value of the aggregate expression level and the aggregate oxidation level, or means for establishing said reference value or threshold value, wherein the reference value or threshold value represents a known diagnosis, prediction and/or prognosis of cancer, or wherein the reference value or threshold value corresponds to the aggregate expression level and the aggregate oxidation level of a group of PTPs at least comprising PTPN2, PTPRF, PTPRK, PTPN12, PTPN7, PTPN1, and PTPN6 in a sample not affected by cancer, or wherein the reference value or threshold value corresponds to the aggregate expression level and the aggregate oxidation level of a group of PTPs at least comprising PTPN2, PTPRF, PTPRK, PTPN12, PTPN7, PTPN1, and PTPN6 in a sample affected by cancer.

20. The kit of claim 17 further comprising:
means for measuring the aggregate expression level of a group of PTPs at least comprising PTPRB, PTPRD, PTPRF, PTPRM, PTPRG, PTPRK, PTPRJ, PTPRC, PTPRA, PTPRE, PTPN1, PTPN2, PTPN3, PTPN6, PTPN7, PTPN9, PTPN11 and PTPN12 in a sample of the subject;
means for measuring the aggregate oxidation level of a group of PTPs at least comprising PTPRB, PTPRD, PTPRF, PTPRM, PTPRG, PTPRK, PTPRJ, PTPRC, PTPRA, PTPRE, PTPN1, PTPN2, PTPN3, PTPN6, PTPN7, PTPN9, PTPN11 and PTPN12 in the sample of the subject; and
a reference value or threshold value of the aggregate expression level and the aggregate oxidation level o, or means for establishing the reference value or threshold value, wherein the reference value or threshold value represents a known diagnosis, prediction and/or prognosis of cancer, or wherein the reference value or threshold value corresponds to the aggregate expression level and the aggregate oxidation level of a group of PTPs at least comprising PTPRB, PTPRD, PTPRF, PTPRM, PTPRG, PTPRK, PTPRJ, PTPRC, PTPRA, PTPRE, PTPN1, PTPN2, PTPN3, PTPN6, PTPN7, PTPN9, PTPN11 and PTPN12 in a sample not affected by cancer, or wherein the reference value or threshold value corresponds to the aggregate expression level and the aggregate oxidation level of a group of PTPs at least comprising PTPRB, PTPRD, PTPRF, PTPRM, PTPRG, PTPRK, PTPRJ, PTPRC, PTPRA, PTPRE, PTPN1, PTPN2, PTPN3, PTPN6, PTPN7, PTPN9, PTPN11 and PTPN12 in a sample affected by cancer.

21. The kit according to claim 17, further comprising:
means for measuring the expression level of one or more individual PTPs in the sample of the subject; and
a reference value or threshold value of the expression value of the one or more individual PTPs or means for establishing the reference value or threshold value, wherein the reference value or threshold value represents a known diagnosis, prediction and/or prognosis of cancer, or wherein the reference value or threshold value corresponds to the expression level of the one or more individual PTPs in a sample not affected by cancer, or wherein the reference value or threshold value corresponds to the expression level of the one or more individual PTPs in a sample affected by cancer.

22. The kit of claim 18, further comprising:
means for measuring the expression level of one or more individual oxidized PTPs in the sample of the subject; and
a reference value or threshold value of the expression level of the one or more individual oxidized PTPs or means for establishing the reference value or threshold value, wherein the reference value or threshold value represents a known diagnosis, prediction and/or prognosis of cancer, or wherein the reference value or threshold value corresponds to the expression level of the one or more individual oxidized PTPs in a sample not affected by cancer, or wherein the reference value or threshold value corresponds to the expression level of the one or more individual oxidized PTPs in a sample affected by cancer.

23. The kit of claim 21, wherein the individual PTPs and/or the individual oxidized PTPs are selected from PTPRB, PTPRD, PTPRF, PTPRM, PTPRG, PTPRK, PTPRJ, PTPRC, PTPRA, PTPRE, PTPN1, PTPN2, PTPN3, PTPN6, PTPN7, PTPN9, PTPN11 and PTPN12.

24. (canceled)

25. The in vitro method according to claim 1, wherein the cancer is selected from hepatocellular carcinoma (HCC), breast cancer, pancreatic cancer, lung cancer, colon cancer, prostate cancer, melanoma, multiple myeloma, lymphoma, and leukemia.

26. The in vitro method according to claim 1, wherein the cancer is hepatocellular carcinoma.

27. The in vitro method according to claim 1, wherein the sample is a tissue sample or a bodily fluid sample.
